(19) United States
(12) Patent Application Publication
(10) Pub. No.: US 2011/0104686 A1
(43) Pub. Date: May 5, 2011

(54) RAPID DETECTION OF MYCOPLASMA CONTAMINATION IN CELL CULTURE SAMPLES

(75) Inventors: Claudia Litterst, San Francisco, CA (US); Luis A. Ugozzoli, San Rafael, CA (US)

(73) Assignee: Bio-Rad Laboratories, Inc., Hercules, CA (US)

(21) Appl. No.: 12/915,485

(22) Filed: Oct. 29, 2010

Related U.S. Application Data
(60) Provisional application No. 61/256,216, filed on Oct. 29, 2009.

(51) Int. Cl. C12Q 1/68

(52) U.S. Cl. 435/6

(57) ABSTRACT

The present invention provides for methods of detecting mycoplasma, for example in cell culture media.

### CROSS-REFERENCE TO RELATED PATENT APPLICATIONS

The present application claims benefit of priority to U.S. Patent Application No. 61/256,216, filed Oct. 29, 2009, which is incorporated by reference for all purposes.

### BACKGROUND OF THE INVENTION

Mycoplasmas are one of the most frequent cell cultures contaminants (Uphoff, C. C. and Drexler, H. G. (2005) Methods Mol Biol, 290, 13-23). These parasitic or saprophytic bacteria, which lack a cell wall, are classified as Mollicutes. Currently, more than 100 Mollicutes species have been described, but eight species account for over 95% of cell culture contaminations, namely Mycoplasma arginini, M. pirum, M. hominis, M. fermentans, M. salivarium, M. orale, M. hyorhinis and Acholeplasma laidlawii (Tang, J. et al., (2000) J Microbiol Methods, 39, 121-126).

Mycoplasma adsorb to the membranes of cells, deplete nutrients, and alter both proliferation and gene expression of cells, thus leading to unreliable experimental results from infected cultures. Unlike other bacterial contaminants, mycoplasmas are not sensitive to commonly used antibiotics and grow in the cell culture supernatant without causing turbidity of the media. This allows mycoplasma to go unnoticed in tissue cultures unless specific detection methods are used. Therefore, routine testing for Mycoplasma is necessary.

Numerous methods for mycoplasma detection have been described (Drexler, H. G. and Uphoff, C. C. (2002) Cytotechnology, 39, 75-90), including microbial culture, biochemical assays, nucleic acid staining and the polymerase chain reaction (PCR). Each method has advantages and limitations, as discussed elsewhere (Drexler H. G., U. C. C. (2000) The Encyclopedia of Cell Technology. J. Wiley, New York., 1249 pp.). PCR represents a fast, sensitive and high throughput detection method that is routinely used in laboratories. Several primer pairs designed for detection of the most common Mollicutes species have been described in the literature targeting highly conserved DNA sequences, e.g. the 16S and 23S rDNA sequences or the spacer in between the two (Harasawa, R. et al. (1993) Res Microbiol, 144, 489-493; Spaepen, M. et al. (1992) FEMS Microbiol Lett, 78, 89-94; Wirth, M. et al. (1994) Cytotechnology, 16, 67-77; Rawadi, G. and Dussurget, O. (1995) PCR Methods Appl, 4, 199-208). However, because rDNA is highly conserved among bacteria, some of these primers have been shown to detect genealogically related gram-positive bacteria in addition to Mollicutes (Eldering, J. A. et al. (2004) Biologicals, 32, 183-193). Therefore primers targeting single copy genes have been designed including tuf (Stormer, M. et al. (2009) Int J Med Microbiol, 299, 291-300) and the rpoB gene (Kong, H. et al. (2007) Appl Microbiol Biotechnol, 77, 223-232; Palgi, J. et al. (2007) United States patent no. 20070243530). In recent years, primers and PCR conditions have been adapted to be used in real-time PCR (Stormer, M. et al. (2009) Int J Med Microbiol, 299, 291-300; Ishikawa, Y. et al., (2006) In Vitro Cell Dev Biol Anim, 42, 63-69; Harasawa, R. et al. (2005) Microbiol Immunol, 49, 859-863; Schmitt, M. and Pawlita, M. (2009) Nucleic Acids Res.), which provides higher sample throughput, speed and sensitivity. The use of DNA binding dyes, such as Eva Green (Mao, F. et al. (2007) BMC Biotechnol, 7, 76), allow real-time detection and can also be followed by melt curve analysis. The melt curve analysis allows confirmation that the observed amplification is due to the correct target.

### BRIEF SUMMARY OF THE INVENTION

The present invention provides methods of detecting mycoplasma in a cell culture media. In some embodiments, the method comprises:

obtaining an aliquot of cell culture media;
performing a real-time nucleic acid amplification reaction with a DNA polymerase to amplify a mycoplasma nucleic acid, if present, in the aliquot wherein:

nucleic acids in the aliquot are not further purified, and

the amplification reaction comprises an intercalating fluorescent dye that produces a fluorescent signal in the presence of double stranded DNA; and

detecting a melting temperature of an amplification product of the amplification reaction, wherein the presence of an amplification product indicates the presence of mycoplasma in the cell culture.

In some embodiments, the amplification reaction is capable of amplifying any (i.e., all present) of Mycoplasma arginini, M. pirum, M. hominis, M. fermentans, M. salivarium, M. orale, M. hyorhinis and Acholeplasma laidlawii, if present in the aliquot.

In some embodiments, the mycoplasma nucleic acid comprises a portion of at least 50 nucleotides of a rpoB gene. In some embodiments, the performing step comprises amplifying the portion with a first degenerate primer comprising or consisting of:

### GAAGAWATGCCWTATTTAGAAGATGG (SEQ ID NO:1); and

a second degenerate primer comprising or consisting of:

### CCRTTTTGACTYTTWCCACCMAGTGGTTGTTG (SEQ ID NO:2),

wherein W represents A or T, Y represents C or T, R represents A or G, and M represents A or C.

In some embodiments, the reaction contains no more primers than one forward primer and one reverse primer.

In some embodiments, the detecting step further comprises nucleotide sequencing the amplification product and correlating the determined nucleotide sequence to nucleotide sequences of different mycoplasma species, thereby determining the identity of the mycoplasma.

In some embodiments, the amplification reaction does not comprise a detectably-labeled oligonucleotide.

In some embodiments, the polymerase is linked to a sequence non-specific DNA binding domain. In some embodiments, the sequence non-specific DNA binding domain is an Sso7 DNA binding domain.

In some embodiments, the aliquot comprises a sufficient amount of an amplification inhibitor to inhibit activity of Taq polymerase by at least 10%. In some embodiments, the inhibitor is selected from the group consisting of cell debris, cell waste products (e.g., polysaccharides or proteins), and fetal bovine serum or an amplification inhibitor component thereof.

In some embodiments, the amplification reaction comprises a sufficient amount of an osmolyte and/or heparin to improve efficiency of the amplification reaction. In some embodiments, the osmolyte is selected from the group consisting of sarcosine, trimethylamine N-oxide (TMAO), dimethylsulfoniopropionate, and trimethylglycine.

The present invention also provides for a method of detecting mycoplasma in a sample (including but not limited to in a cell culture media). In some embodiments, the method comprises,

obtaining an aliquot of a sample (including but not limited to cell culture media);
performing a nucleic acid amplification reaction with a DNA polymerase to amplify a mycoplasma nucleic acid, if present, in the aliquot wherein:
(a) the performing step comprises amplifying the portion with a first and second primer, comprising a first degenerate primer comprising or consisting of:

### GAAGAWATGCCWTATTTAGAAGATGG (SEQ ID NO:1); and

a second degenerate primer comprising or consisting of:
CCRTTTTGACTYTTWCCACCMAGTGGTTGTTG (SEQ ID NO:2), wherein W represents A or T, Y represents C or T, R represents A or G, and M represents A or C; and
(b) detecting the presence or absence of an amplification product of the amplification reaction, wherein the presence of an amplification product indicates the presence of mycoplasma in the sample.

In some embodiments, the amplification reaction is monitored in real-time. In some embodiments, the amplification reaction comprises an intercalating fluorescent dye that produces a fluorescent signal in the presence of double stranded DNA at least twice that produced in the presence of single-stranded DNA only.

In some embodiments, the first and second primers consist of the following degenerate sequences, respectively:

| | |
|---|---|
| GAAGAWATGCCWTATTTAGAAGATGG; | (SEQ ID NO: 1) |
| and | |
| CCRTTTTGACTYTTWCCACCMAGTGGTTGTTG | (SEQ ID NO: 2) |

In some embodiments, nucleic acids in the aliquot are not further purified.

In some embodiments, the reaction contains no more than one forward and one reverse primer designed to hybridize to an rpoB gene.

In some embodiments, the detecting step further comprises nucleotide sequencing the amplification product and correlating the determined nucleotide sequence to nucleotide sequences of different mycoplasma species, thereby determining the identity of the mycoplasma.

In some embodiments, the amplification reaction does not comprise a detectably-labeled oligonucleotide.

In some embodiments, the polymerase is linked to a sequence non-specific DNA binding domain. In some embodiments, the sequence non-specific DNA binding domain is an Sso7 DNA binding domain.

In some embodiments, the amplification reaction comprises a sufficient amount of an osmolyte and/or heparin to improve efficiency of the amplification reaction. In some embodiments, the osmolyte is selected from the group consisting of sarcosine, trimethylamine N-oxide (TMAO), dimethylsulfoniopropionate, and trimethylglycine.

The present invention also provides kits for amplifying mycoplasma DNA, if present, from cell culture media. In some embodiments, the kit comprises:

a first degenerate primer comprising GAAGAWATGCCWTATTTAGAAGATGG (SEQ ID NO:1); and
a second degenerate primer comprising CCRTTTTGACTYTTWCCACCMAGTGGTTGTTG (SEQ ID NO:2),
wherein W represents A or T, Y represents C or T, R represents A or G, and M represents A or C.

In some embodiments, the first primer consists of GAAGAWATGCCWTATTTAGAAGATGG (SEQ ID NO:1); and

the second primer consists of CCRTTTTGACTYTTWCCACCMAGTGGTTGTTG (SEQ ID NO:2).

In some embodiments, the kit further comprises at least one or more of the following:

a polymerase;
an intercalating fluorescent dye;
a positive control polynucleotide comprising a polynucleotide that can be amplified by a polymerase primed by SEQ ID NO:1 or SEQ ID NO:2 (i.e., wherein the polynucleotide comprises sequences sufficiently complementary (optionally 100% complementary) such that primers consisting of SEQ ID NO:1 and SEQ ID NO:2, respectively, can amplify an intervening section of the polynucleotide under amplification conditions).

In some embodiments, the kit further comprises a positive control sample comprising a nucleic acid comprising at least 50 contiguous nucleotides of a Mycoplasma rpoB gene.

In some embodiments, the kit further comprises an osmolyte and/or heparin. In some embodiments, the osmolyte is selected from the group consisting of sarcosine, trimethylamine N-oxide (TMAO), dimethylsulfoniopropionate, and trimethylglycine.

In some embodiments, the polymerase is linked to a sequence non-specific DNA binding domain. In some embodiments, the sequence non-specific DNA binding domain is an Sso7 DNA binding domain.

### DEFINITIONS

The terms “oligonucleotide” or “polynucleotide” or “nucleic acid” interchangeably refer to a polymer of monomers that can be corresponded to a ribonucleic acid (RNA) and deoxyribonucleic acid (DNA) polymer, or analog thereof. This includes polymers of nucleotides such as RNA and DNA, as well as modified forms thereof, peptide nucleic acids (PNAs), locked nucleic acids (LNA™), and the like. In certain applications, the nucleic acid can be a polymer that includes multiple monomer types, e.g., both RNA and DNA subunits.

A nucleic acid is typically single-stranded or double-stranded and will generally contain phosphodiester bonds, although in some cases, as outlined herein, nucleic acid analogs are included that may have alternate backbones, including, for example and without limitation, phosphoramide (Beaucage et al. (1993) Tetrahedron 49(10):1925 and the references therein; Letsinger (1970) J. Org. Chem. 35:3800; Sprinzl et al. (1977) Eur. J. Biochem. 81:579; Letsinger et al. (1986) Nucl. Acids Res. 14: 3487; Sawai et al. (1984) Chem. Lett. 805; Letsinger et al. (1988) J. Am. Chem. Soc. 110:4470; and Pauwels et al. (1986) Chemica Scripta 26:1419), phosphorothioate (Mag et al. (1991) Nucleic Acids Res. 19:1437 and U.S. Pat. No. 5,644,048), phosphorodithioate (Briu et al. (1989) J. Am. Chem. Soc. 111:2321), O-methylphosphoroamidite linkages (Eckstein, Oligonucleotides and Analogues: A Practical Approach, Oxford University Press (1992)), and peptide nucleic acid backbones and linkages (Egholm (1992) J. Am. Chem. Soc. 114:1895; Meier et al. (1992) Chem. Int. Ed. Engl. 31:1008; Nielsen (1993) Nature 365:566; and Carlsson et al. (1996) Nature 380:207), which references are each incorporated by reference. Other analog nucleic acids include those with positively charged backbones (Denpcy et al. (1995) Proc. Natl. Acad. Sci. USA 92:6097); non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook; Mesmaeker et al. (1994) Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook, which references are each incorporated by reference. Nucleic acids containing one or more carbocyclic sugars are also included within the definition of nucleic acids (Jenkins et al. (1995) Chem. Soc. Rev. pp 169-176, which is incorporated by reference). Several nucleic acid analogs are also described in, e.g., Rawls, C & E News Jun. 2, 1997 page 35, which is incorporated by reference. These modifications of the ribose-phosphate backbone may be made to facilitate the addition of additional moieties such as labeling moieties, or to alter the stability and half-life of such molecules in physiological environments.

In addition to naturally occurring heterocyclic bases that are typically found in nucleic acids (e.g., adenine, guanine, thymine, cytosine, and uracil), nucleic acid analogs also include those having non-naturally occurring heterocyclic or other modified bases, many of which are described, or otherwise referred to, herein. In particular, many non-naturally occurring bases are described further in, e.g., Seela et al. (1991) Helv. Chim. Acta 74:1790, Grein et al. (1994) Bioorg. Med. Chem. Lett. 4:971-976, and Seela et al. (1999) Helv. Chim. Acta 82:1640, which are each incorporated by reference. To further illustrate, certain bases used in nucleotides that act as melting temperature (Tm) modifiers are optionally included. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, entitled “SYNTHESIS OF 7-DEAZA-2′-DEOXYGUANOSINE NUCLEOTIDES,” which issued Nov. 23, 1999 to Seela, which is incorporated by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytosine; 5-fluorocytosine; 5-chlorocytosine; 5-iodocytosine; 5-bromocytosine; 5-methylcytosine; 5-propynylcytosine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like. The primers of the invention, for example, can comprise one, two, or more of the above-described non-natural nucleotides.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence, which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, designated region as measured using one of the following sequence comparison algorithms or by manual alignment and visual inspection, or across the entire sequence where not indicated.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. Unless indicated otherwise, default parameters can be assumed. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

A “thermostable” polymerase is used as generally used in the art and refers to a polymerase that substantially retains activity at elevated temperatures (e.g., 90° C.), for example such that the polymerase is effective for use in multiple cycles of a PCR reaction.

### BRIEF DESCRIPTION OF THE DRAWINGS

FIG. 1: Design of universal primers for detection of rpoB gene from mollicutes. View of primer binding regions and primer sequences. The following mixed base pairs were used: K: G, T: W: A, T: R: A, G: Y: C, T. The sequences displayed are: Forward primers; M. pirum (P) (SEQ ID NO:7), A. laidlawii (L) (SEQ ID NO:9), M. arginini (A) (SEQ ID NO:11), M. orale (O) (SEQ ID NO:13), M. salivarium (S) (SEQ ID NO:15), M. hominis (H) (SEQ ID NO:17), M. fermentans (F) (SEQ ID NO:19), M. hyorhinis (Hy) (SEQ ID NO:21), and Forward consensus (SEQ ID NO:23). Reverse primers; M. pirum (P) (SEQ ID NO:8), A. laidlawii (L) (SEQ ID NO:10), M. arginini (A) (SEQ ID NO:12), M. orale (O) (SEQ ID NO:14), M. salivarium (S) (SEQ ID NO:16), M. hominis (H) (SEQ ID NO:18), M. fermentans (F) (SEQ ID NO:20), M. hyorhinis (Hy) (SEQ ID NO:22), and Reverse consensus (SEQ ID NO:24).

FIG. 2: Specificity of rpoB PCR. A) 20 pg of genomic DNA from the indicated mycoplasma species was amplified using the rpoB primers. The Figure shows the amplification plots and melt curve analysis. B) The PCR products from A were analyzed on a 3% Agarose gel. C) 1 ng of genomic DNA from Rat, Human, Mouse, Bacillus subtilis, Lactobacillus casei, Candida albicans, E. coli along with 1 pg of genomic DNA from M. fermentans was analyzed by PCR as described in A).

FIG. 3: Sensitivity of rpoB PCR. Serial dilutions from 100 pg-1 fg genomic DNA from A. laidlawii (A) and M. fermentans (B) were subjected to real-time PCR amplification using the rpoB primers indicated in FIG. 1B and the SsoFast EvaGreen Supermix. Amplification plots, melt curves and standard curves of triplicate reactions are shown.

FIG. 4: Detection of mycoplasmas in crude samples by the real-time PCR assay. Reactions were set up as indicated in Table 2. Conditioned media from various contaminated and not infected cell lines along with fresh media and water was added at 10% final concentration. The amplification plot and melt peak analysis of the triplicate PCR reactions are shown.

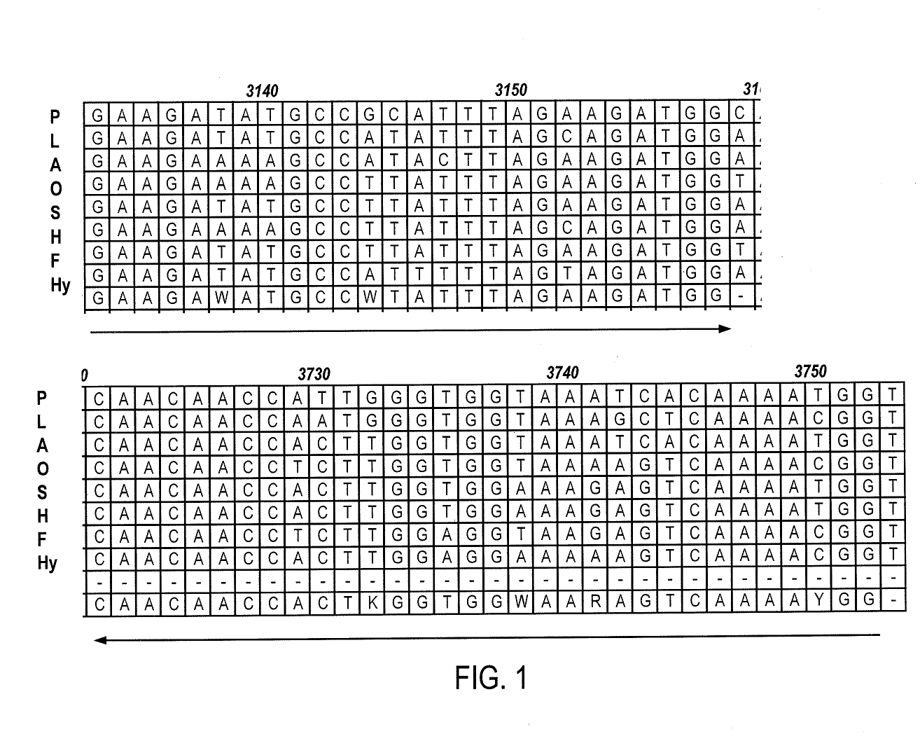

Table 1: Effect of common media formulations on the rpoB PCR amplification by our real-time PCR assay. PCR reactions were set up using the primers shown in FIG. 1B along with purified genomic mycoplasma DNA and SsoFast EvaGreen Supermix in the presence or absence (water) of various tissue culture media (10% final concentration) as indicated. The reactions were performed in triplicate and the average Ct and standard deviations are shown.

Table 2: Effect of conditioned media from aged cultures on the amplification of the rpoB gene by our real-time PCR assay. Reactions were set up as indicated in Table 1. Conditioned media (cond) from various cell lines that were grown to confluency for more than 4 days was added at 10% final concentration as indicated. Average Ct and standard deviations from triplicates are shown.

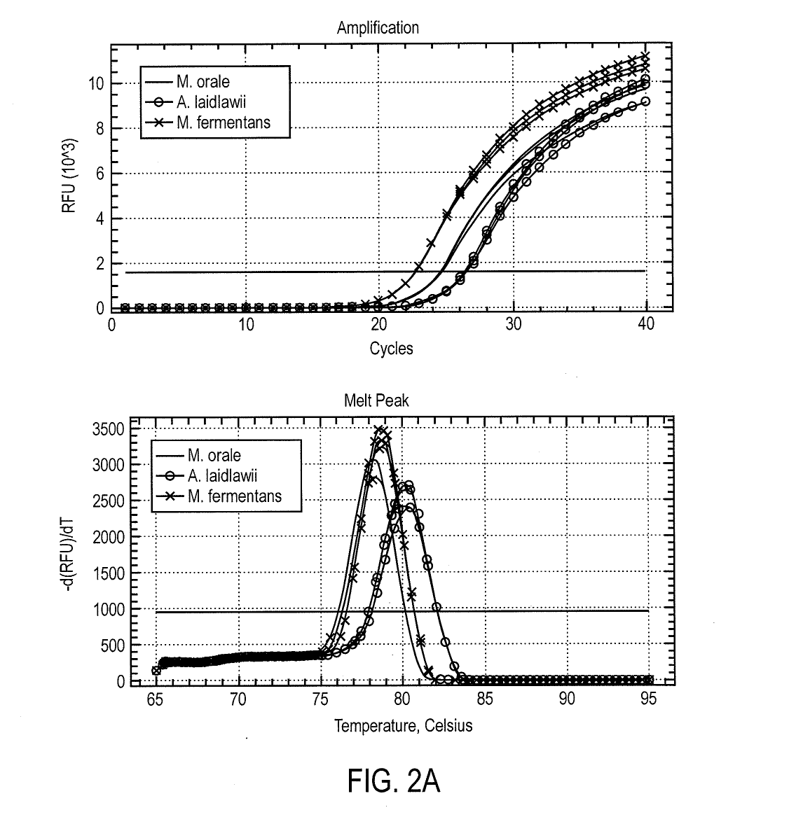

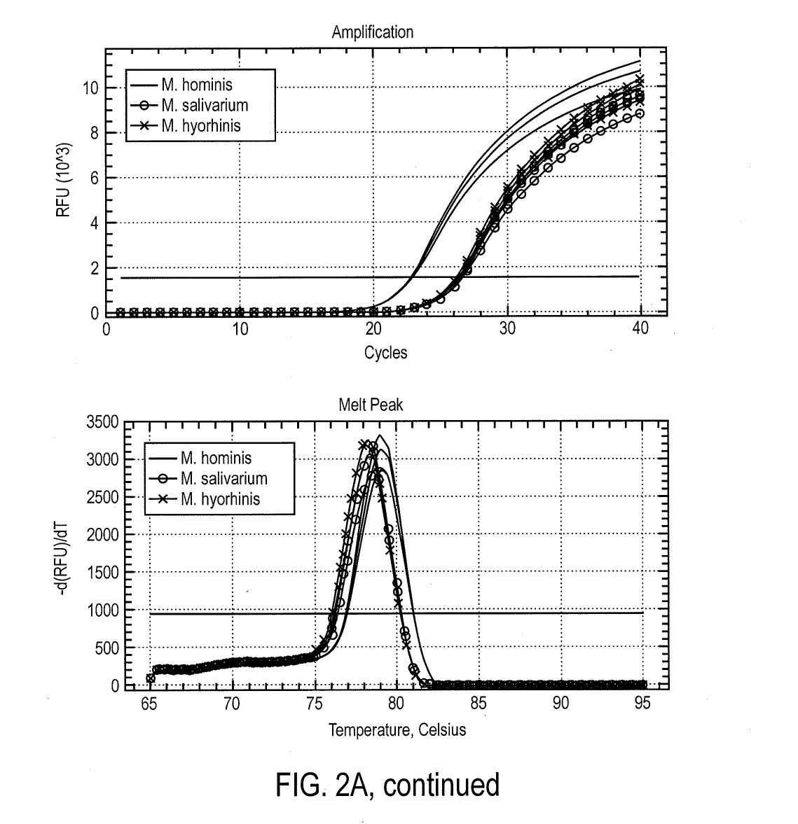

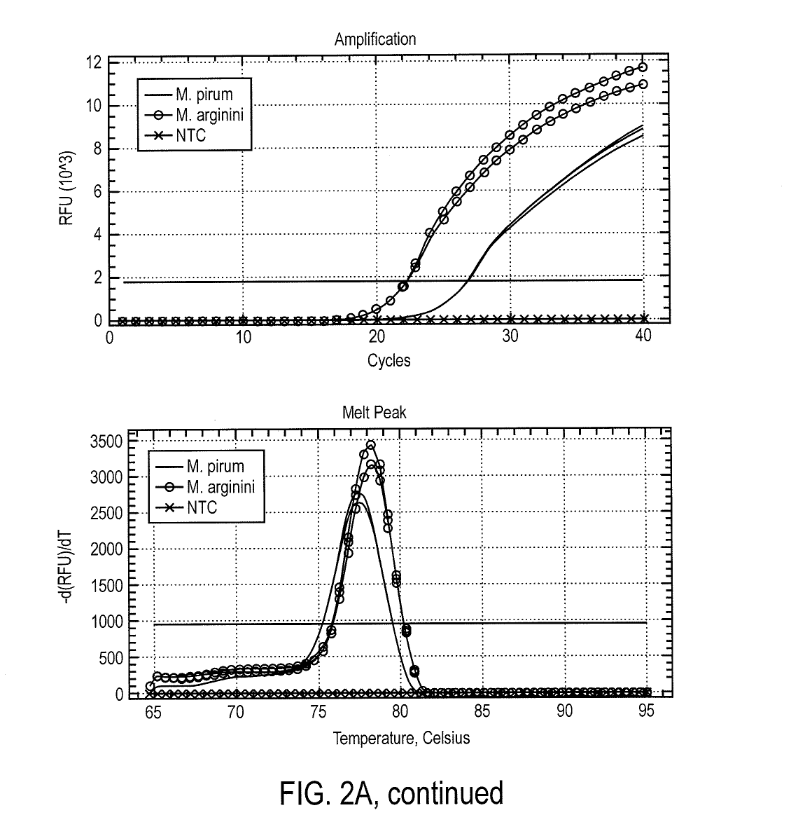

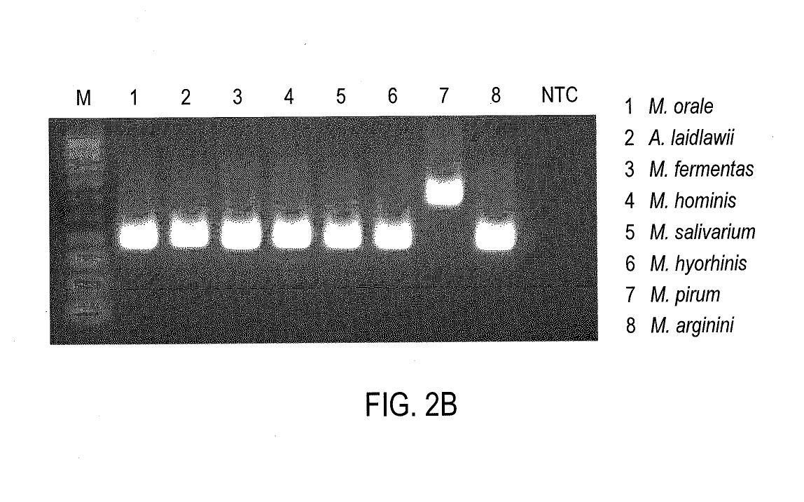

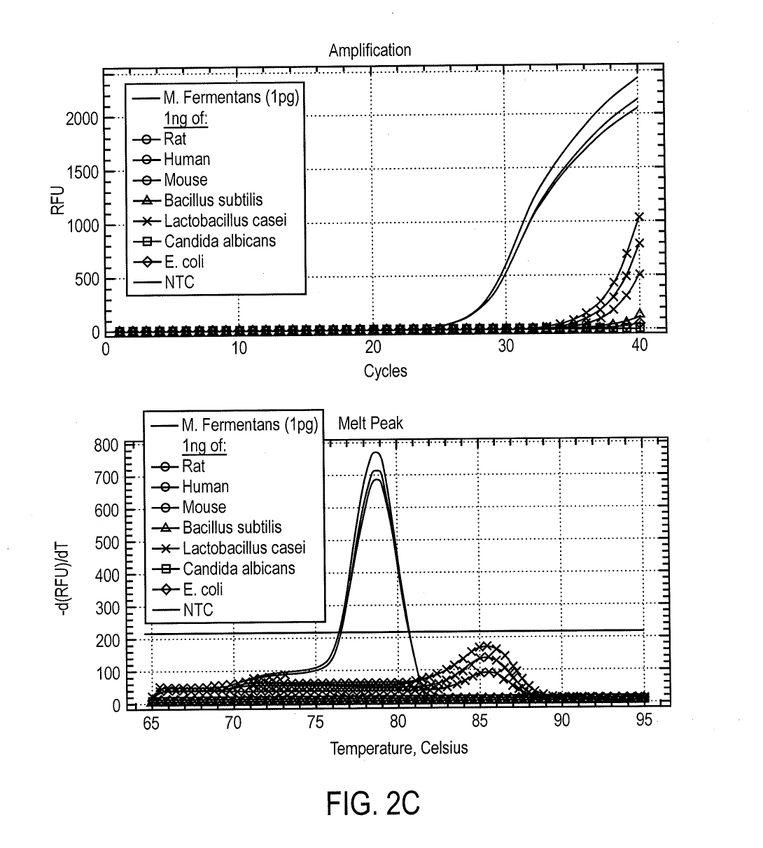

Table 3: Detection of mycoplasmas in crude samples. Shown is the average Ct as well as standard deviations and the major melt peak from triplicate reactions described in FIG. 2. Samples were determined to be positive when the melt peak was between 77 and 79° C.

### DETAILED DESCRIPTION

### I. Introduction

The present invention is based, in part, on the surprising discovery that polymerase chain reaction (PCR) can be used to detect a large number of different mycoplasma species in unpurified culture media samples with minimal or no inhibition. The invention is of particular use in combination with polymerase enzymes that are resistant to polymerase inhibitors commonly found in cell culture media. Further, the inventors have identified a particular primer set that allows for amplification and detection of a large number of mycoplasma species with a single primer pair. The primers, in combination with an inhibitor-resistant polymerase, creates a powerful combination that allows for rapid and efficient detection of mycoplasma in cell culture media.

### II. Primers of the Invention

Any target nucleic acids of mycoplasma origin can be used as targets for an amplification-based approach of the invention. Preferably, the mycoplasma nucleic acids are not common in other organisms. For example, in some embodiments, a particular target nucleic acid is not present in mammalian cells (e.g., human, rat, mouse, etc.) or at least is not amplified during the method sufficiently to alter interpretation of the assay for the presence or absence of mycoplasma. While it is generally desirable that the primer of the invention also not significantly amplify non-mycoplasma bacterial nucleic acids, this is not always possible or necessary. In some embodiments, if nucleic acids from commonly-occurring bacterial species are also amplifiable with the primers of the invention, in some embodiments they can be distinguished from mycoplasma amplicons by melting temperature or other detectable characteristic (including but not limited to determining the amplicon length or performing nucleotide sequencing) of the amplicons.

Exemplary nucleic acid targets for detection in mycoplasma species include, but are not limited to the tuf gene (see, e.g., Stormer et al., Int. J. Med. Microbiol., 299:291-300 (2009)), the P1 gene (see, e.g., U.S. Pat. No. 6,277,582), ribosomal RNa (rRNA) structural genes and spacer regions including but not limited to the 16S gene and/or spacer region. In some embodiments, one or more primer pair is used to amplify at least a portion (e.g., at least 20, 50, 100, base pairs, etc.) of the rpoB gene. One exemplary primer set is described in, e.g., Kong et al., Appl. Microbiol. Biotechnol. 77:223-232 (2007), incorporated by reference.

The inventors have discovered that a particular primer set significantly improves over previous Mycoplasma detection systems by allowing for rapid detection of Mycoplasma arginini, M. pirum, M. hominis, M. fermentans, M. salivarium, M. orale, M. hyorhinis and Acholeplasma laidlawii, which represent 95% of all cell culture mycoplasma contaminations, with only one forward and one reverse primer. The primers of the invention target the rpoB gene. The primers do not amplify mammalian nucleic acids or most other bacterial nucleic acids, allowing for a tentative conclusion of Mycoplasma presence by simple detection of signal, i.e., detection of the generation of the amplification product. Signal can be detected, for example, by detecting the melting temperature of the amplification product. The melting temperature is useful, for example, for distinguishing generation of the amplification product from generation of, e.g., primer dimers or other artifacts.

The target region of the primers are set forth in FIG. 1, with the area of primer hybridization and directionality of the primers shown with arrows. The degenerate primers used in the examples are as follows:

| | |
|---|---|
| GAAGAWATGCCWTATTTAGAAGATGG; | (SEQ ID NO: 1) |
| and | |
| CCRTTTTGACTYTTWCCACCMAGTGGTTGTTG, | (SEQ ID NO: 2) |

wherein W represents A or T, Y represents C or T, R represents A or G, and M represents A or C. “Degenerate” primers refer to mixtures of similar, but not identical, primers. Thus, for the above primers, a forward primer comprising SEQ ID NO:1 involves a mixture of related primers in which W, Y, R, and M represent the one or more positions at which the related primers differ. In some embodiments of the invention, the forward and reverse primers consist, or consist essentially of SEQ ID NO:1 and SEQ ID NO:2, respectively. One can generate degenerate primer mixtures for example using many modern oligonucleotide synthesis machines.

In some embodiments of the invention, the only primers designed to hybridize to a Mycoplasma rpoB gene, or optionally to any rpoB gene, are those comprising or consisting of SEQ ID NO:1 and SEQ ID NO:2. Thus, for example, in some embodiments, the only forward primer in the amplification reaction comprises or consists of SEQ ID NO:1 and the only reverse primer in the amplification reaction comprises or consists of SEQ ID NO:2.

In some embodiments, the primers of the invention will comprise SEQ ID NO:1 and SEQ ID NO:2 (forward and reverse respectively) and will comprise one or more additional nucleotides and/or other moieties (e.g., labels, tags, biotin, etc.). The one or more nucleotides can include nucleotides complementary to a Mycoplasma rpoB gene (as aligned with the remainder of the primer) and/or additional nucleotides (e.g., containing a restriction enzyme recognition sequence, etc.) not complementary to the target, e.g., at the 5′ end of the primer.

As noted above, in some embodiments, the amplification product is nucleotide sequenced. This can be useful, for example, in situations where one desires to know the exact species of Mycoplasma and/or to confirm that the contamination is not due to amplification of bacterial DNA. In embodiments where the rpoB gene is amplified, it has been found that use of either or both of sequencing primers: TGC ATT TTG TCA TCA ACC ATG TG (SEQ ID NO:5) and/or CCT TCA CGT ATG AAC AT (SEQ ID NO:6) allow for differentiation of the species if Mycoplasma rpoB DNA is amplified. It is possible to distinguish species with sequence runs of at least about 50 nucleotides from each primer, though it is generally helpful to allow the sequencing run to go to completion (e.g., 300-500 nucleotides). For example, nucleotide sequencing with SEQ ID NO:5 as the sequencing primer allows one to identify M. pirum, M. arginini, M. orale, M. salivarium, M. hominis, M. fermentans, and M. hyorhinis. Nucleotide sequencing with SEQ ID NO:6 as the sequencing primer allows one to identify A. laidlawii, M. arginini, M. salivarium, M. hominis, M. fermentans, and M. hyorhinis.

### III. Polymerase Chain Reaction

The invention can be performed using any sort of nucleic acid amplification method.

In some embodiments, the methods of the invention are performed with the polymerase chain reaction. At a basic level, the methods of the invention involving PCR comprise amplifying at least a portion of a target mycoplasma nucleic acid present in a cell culture and then detecting the presence or absence of the resulting amplicon. Optionally, following PCR, one can detect and/or analyze (e.g., determine physical characteristics such as length, ability to migrate in a gel, determine the melting temperature, determine the nucleotide sequence, etc.) the resulting amplicon.

In some embodiments, the PCR is quantitative PCR in which the accumulation of amplicon is monitored in “real time” (i.e., continuously, e.g., once per cycle—rather than only following the completion of amplification). Quantitative amplification methods (e.g., quantitative PCR or quantitative linear amplification) involve amplification of an nucleic acid template, directly or indirectly (e.g., determining a Ct value) determining the amount of amplified DNA, and then calculating the amount of initial template based on the number of cycles of the amplification. Amplification of a DNA locus using reactions is well known (see U.S. Pat. Nos. 4,683,195 and 4,683,202; PCR PROTOCOLS: A GUIDE TO METHODS AND APPLICATIONS (Innis et al., eds, 1990)). Typically, PCR is used to amplify DNA templates. However, alternative methods of amplification have been described and can also be employed, as long as the alternative methods amplify intact DNA to a greater extent than the methods amplify cleaved DNA. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol. Biotechnol. 20(2):163-79 (2002).

In some embodiments, quantitative amplification is based on the monitoring of the signal (e.g., fluorescence of a probe) representing copies of the template in cycles of an amplification (e.g., PCR) reaction. In the initial cycles of the PCR, a very low signal is observed because the quantity of the amplicon formed does not support a measurable signal output from the assay. After the initial cycles, as the amount of formed amplicon increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., Heid et al. Genome Methods 6:986-94 (1996) with reference to hydrolysis probes.

One method for detection of amplification products is the 5′-3′ exonuclease “hydrolysis” PCR assay (also referred to as the TaqMan™ assay) (U.S. Pat. Nos. 5,210,015 and 5,487,972; Holland et al., PNAS USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the “TaqMan™ probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer, Nature Biotech. 14:303-309 (1996), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in the open conformation and fluorescence is detected, while those that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of a nucleic acids are also known. For example, some methodologies employ one or more probe oligonucleotides that are structured such that a change in fluorescence is generated when the oligonucleotide(s) is hybridized to a target nucleic acid. For example, one such method involves is a dual fluorophore approach that exploits fluorescence resonance energy transfer (FRET), e.g., LightCycler™ hybridization probes, where two oligo probes anneal to the amplicon. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., Nature Biotechnology 17:804-807, 1999, and U.S. Pat. No. 6,326,145), Sunrise™ (or Amplifluor™) probes (e.g., Nazarenko et al., Nuc. Acids Res. 25:2516-2521, 1997, and U.S. Pat. No. 6,117,635), and probes that form a secondary structure that results in reduced signal without a quencher and that emits increased signal when hybridized to a target (e.g., Lux probes™).

In some embodiments, the PCR reaction mixture does not include a labeled probe oligonucleotide. For example, the reaction mixture lacks a Taqman or other labeled oligonucleotide probe for monitoring real-time or endpoint accumulation of the amplicon. In some of these embodiments, an intercalating fluorescent dye is included. In some embodiments, the intercalating dye changes signal (increases or decreases) when bound to double stranded nucleic acids compared to signal stranded nucleic acids. Exemplary agents include SYBR GREEN™, SYBR GOLD™, and EVAGREEN™. Since these agents are not template-specific, it is assumed that the signal is generated based on template-specific amplification. This can be confirmed by monitoring signal as a function of temperature because melting point of template sequences will generally be much higher than, for example, primer-dimers, etc.

A number of components of a PCR reaction are well known and can be determined readily by a skilled artisan. In certain aspects, it may be desirable to include an additional compound as an additive to improve efficiency in amplification reactions, such as qPCR. For example, there may be situations in which a polymerase of the invention that lacks exonuclease activity exhibits low efficiency for certain targets when used in a formulation that includes certain binding dyes (such as, in one non-limiting example, an EvaGreen DNA binding dye) or in the presence of certain amplification inhibitors. Such low efficiency may in some embodiments be a result of delay in Ct values associated with low input DNA concentrations. Methods for measuring efficiency of a particular reaction are known in the art.

In some embodiments, an osmolyte may be included in an amplification reaction of the invention to improve efficiency. See, e.g., WO2010/080910, incorporated by reference. Members of the osmolyte family have been shown to improve the thermal stability of proteins (Santoro, Biochemistry, 1992) as well as decrease DNA double helix stability (Chadalavada, FEBS Letters, 1997). Osmolytes of use in the present invention may include without limitation sarcosine, trimethylamine N-oxide (TMAO), dimethylsulfoniopropionate, and trimethylglycine. Sarcosine is chemically similar to betaine, a chemical which has been shown to improve conventional PCR (Henke, Nucleic Acids Research, 1997).

In conventional uses of osmolytes, the stabilizing effects of such compounds are generally observed at relatively high concentrations (>1M). However, in methods of the present invention, millimolar concentrations of osmolytes have been found to be effective for improving the reaction efficiency of amplification reactions such as qPCR. See, e.g., WO2010/080910, incorporated by reference. Without being bound by a mechanism of action, it is possible that the improvement in efficiency is the result of improvement of the Ct values for the reactions that contain low DNA template concentration. In some embodiments, concentrations of about 100 to about 1000 mM of osmolytes are used in methods and kits of the present invention. In still further embodiments, concentrations of about 50 to about 700, about 100 to about 600, about 150 to about 500, about 200 to about 400 mM, or about 300 to about 350 mM osmolytes are used in methods and kits of the invention. In some embodiments, the osmolyte used in methods and kits of the invention is sarcosine. Indeed, it has been found that addition of sarcosine improved the efficiency of the amplification reaction as compared to control comprising water.

In some embodiments, particularly in the amplification of low-copy target nucleic acids or in the presence of amplification inhibitors, efficiency decreases due to the binding of polymerase to non-primed double-stranded nucleic acid targets. Binding of the polymerase to the double-stranded targets will prevent those targets from denaturation, hybridizing to primers, and undergoing an amplification reaction. To improve the specificity of the polymerase for primed templates, in some embodiments methods and kits of the invention utilize heparin. See, e.g., WO2010/080910, incorporated by reference. Heparin molecules, which are negatively charged, can be included in the reaction mixture to mimic the electrostatic property of double stranded nucleic acids. The addition of heparin can, without being limited to a mechanism of action, prevent excess polymerase from binding to the double-stranded template until a single-stranded primed-template becomes available. In some exemplary embodiments, heparin is used in methods and kits of the invention at concentrations of about 50 to about 750 pg/μl. In further exemplary embodiments, heparin is used in methods and kits of the invention at concentrations of about 75 to about 700, about 100 to about 600, about 125 to about 500, about 150 to about 400, about 175 to about 300, or about 200 to about 250 pg/μl.

### IV. Polymerases

DNA polymerases useful in the present invention can be any polymerase capable of replicating a DNA molecule. Exemplary DNA polymerases are thermostable polymerases, which are especially useful in PCR. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus woesei (Pwo) and other species of the Pyrococcus genus, Bacillus sterothermophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof.

In some embodiments, the polymerase enzyme is a hybrid polymerase comprising a polymerase domain and a DNA binding domain. Such hybrid polymerases are known to show an increased processivity. See e.g., U.S. Patent Application Publication Nos. 2006/005174; 2004/0219558; 2004/0214194; 2004/0191825; 2004/0081963; 2004/0002076; 2003/0162173; 2003/0148330; 2003/0138830 and U.S. Pat. Nos. 6,627,424 and 7,445,898, each of which is hereby incorporated by reference in its entirety for all purposes and in particular for all teachings related to polymerases, hybrid/chimeric polymerases, as well as all methods for making and using such polymerases.

In one aspect, the present invention provides hybrid polymerases that lack 3′-5′ exonuclease activity. In one embodiment, such hybrid polymerases comprise a double point mutation in the polymerase domain that provides this exonuclease deficiency. In a specific embodiment, hybrid polymerases of the invention comprise double point mutation D141A/E143A in the polymerase domain.

In a further embodiment, hybrid polymerases of the invention are encoded by a nucleotide sequence according to SEQ ID NO: 3. In a still further embodiment, hybrid polymerases of the invention are encoded by a nucleotide sequence that has about 50%, 55%, 60%, 65%, 70%, 75%, 80%, 85%, 90%, 91%, 92%, 93%, 94%, 95%, 96%, 97%, 98%, and 99% sequence identity to SEQ ID NO:3.

In a further embodiment, hybrid polymerases of the invention have an amino acid sequence according to SEQ ID NO:4. In a still further embodiment, hybrid polymerases of the invention have an amino acid sequence with about 50%, 55%, 60%, 65%, 70%, 75%, 80%, 85%, 90%, 91%, 92%, 93%, 94%, 95%, 96%, 97%, 98%, and 99% sequence identity to SEQ ID NO:4.

In some embodiments, the binding domain of hybrid polymerases of the invention are from a thermostable organism and provides enhanced activity at higher temperatures, e.g., temperatures above 45° C. For example, Sso7d and Sac7d are small (about 7 kd MW), basic chromosomal proteins from the hyperthermophilic archaeabacteria Sulfolobus solfataricus and S. acidocaldarius, respectively (see, e.g., Choli et al., Biochimica et Biophysica Acta 950:193-203, 1988; Baumann et al., Structural Biol. 1:808-819, 1994; and Gao et al, Nature Struc. Biol. 5:782-786, 1998). These proteins bind DNA in a sequence-independent manner and when bound, increase the Tm of DNA by up to 40° C. under some conditions (McAfee et al., Biochemistry 34:10063-10077, 1995). These proteins and their homologs are often used as the sequence-non-specific DNA binding domain in improved polymerase fusion proteins. Sso7d, Sac7d, Sac7e and related sequences (referred to herein as “Sso7 sequences” or “Sso7 domains”) are known in the art (see, e.g., accession numbers (P39476 (Sso7d) (SEQ ID NO:25); P13123 (Sac7d) (SEQ ID NO:26); and P13125 (Sac7e) (SEQ ID NO:27)). These sequences typically have at least 75% or greater, of 80%, 85%, 90%, or 95% or greater, amino acid sequence identity. For example, an Sso7 protein typically has at least 75% identity to an Sso7d sequence.

In further embodiments, hybrid polymerases of use in the present invention are described for example in U.S. Patent Application Publication Nos. 2006/005174; 2004/0219558; 2004/0214194; 2004/0191825; 2004/0081963; 2004/0002076; 2003/0162173; 2003/0148330; 2003/0138830 and U.S. Pat. Nos. 6,627,424 and 7,445,898, each of which is hereby incorporated by reference in its entirety for all purposes and in particular for all teachings related to polymerases, hybrid/chimeric polymerases, as well as all methods for making and using such polymerases. Examples of hybrid polymerase proteins and methods of generating hybrid proteins are also disclosed in WO2004011605, which is hereby incorporated by reference in its entirety for all purposes, and in particular for all teachings related to generating hybrid proteins.

### V. Sample Preparation

As explained above and in the examples, one aspect of the present invention is that one does not need to purify nucleic acids from cell cultures, or remove inhibitors present in the cell culture, prior to amplification of the target mycoplasma nucleic acids, if present. Accordingly, the present invention provides for aspects where an aliquot of cell culture media, optionally containing mammalian or other non-prokaryotic cells, are added to an amplification reaction and amplified for the presence of mycoplasma nucleic acids. In short, no sample preparation is required in that an aliquot of cell culture media can be used directly in an amplification, particularly where the rpoB primers described herein are use and/or the stabilizing additives (e.g., osmolytes and/or heparin) and/or a hybrid polymerase comprising a heterologous DNA binding domain is used.

Exemplary non-limiting culture media for animal cells include, but are not limited to DMEM, MEM, RPMI1640, or IMDM. In some embodiments, the cell medium will include fetal calf serum (FCS) or fetal bovine serum (FBS). A number components found in cell cultures can be inhibitory to some polymerases. Inhibitory factors can include, e.g., hemoglobin, lactoferrin and immunoglobulin, as well as for example, cell debris, cell waste products (e.g., polysaccharides or proteins).

### VI. Kits

The present invention also provides kits, e.g., for detecting possible mycoplasma contamination in cell cultures. A kit can optionally include written instructions or electronic instructions (e.g., on a CD-ROM or DVD). In some embodiments, kits of the invention will include a case or container for holding the reagents in the kit, which can be included separately or in combination.

In some embodiments, the kits of the invention will include one or more of:
a first primer (i.e., a degenerate mixture of primers) comprising (or consisting of) GAAGAWATGCCWTATTTAGAAGATGG (SEQ ID NO:1) wherein W represents A or T, Y represents C or T, R represents A or G, and M represents A or C;
a second primer (i.e., a degenerate mixture of primers) comprising (or consisting of) CCRTTTTGACTYTTWCCACCMAGTGGTTGTTG (SEQ ID NO:2), wherein W represents A or T, Y represents C or T, R represents A or G, and M represents A or C;
a polymerase (including but not limited to a hybrid polymerase as described herein);
an intercalating fluorescent dye;
a positive control polynucleotide comprising a polynucleotide that can be amplified by a polymerase primed by SEQ ID NO:1 or SEQ ID NO:2; and/or
an osmolyte and/or heparin.

In some embodiments, the kit further comprises the appropriate buffers for enzymatic activity (e.g., including or excluding Ca⁺⁺, Mg⁺⁺, and/or Mn⁺⁺ as appropriate). In some embodiments, the kit further comprises nucleotides (including but not limited to deoxynucleotides or dideoxynucleotides or analogs thereof).

In some embodiments, the kit further comprises a negative control. In some embodiments, the negative control is water or tissue culture media lacking mycoplasma DNA.

### Examples

The following examples are offered to illustrate, but not to limit the claimed invention.

One major shortcoming of PCR is the sensitivity to PCR inhibitors which are present in clinical and tissue culture samples leading to false negative results (Rossen, L. et al. (1992) Int J Food Microbiol, 17, 37-45; Wilson, I. G. (1997) Appl Environ Microbiol, 63, 3741-3751; Razin, S. (1994) Mol Cell Probes, 8, 497-511). Several inhibitory components derived from blood have been identified, including hemoglobin (Al-Soud, W. A. and Radstrom, P. (2001) J Clin Microbiol, 39, 485-493), lactoferrin (Al-Soud, W. A. and Radstrom, P. (2001) J Clin Microbiol, 39, 485-493) and immunoglobulin (Al-Soud, W. A. et al. (2000) J Clin Microbiol, 38, 345-350), all of which are present in most FBS preparations. Cell debris, various proteins and polysaccharides were also described to inhibit PCR reactions (Harasawa, R. et al. (2005) Microbiol Immunol, 49, 859-863; Wilson, I. G. (1997) Appl Environ Microbiol, 63, 3741-3751). Therefore, PCR protocols generally include a sample preparation step to purify genomic DNA from mycoplasma prior to PCR. The sample preparation step is not only time-consuming but also a source for sample loss and errors.

In order to generate an enzyme with higher tolerance to PCR inhibitors, a new polymerase containing a 63 amino acid dsDNA-binding protein of Sso7d that exhibits no sequence preference has been designed (Wang, Y., United States Patent No. 2004/0081963; Wang, Y. et al., United States Patent No. 2004/0002076; Vander Horn, P. B. et al., United States Patent No. 2004/0219558). Sso7d gives the polymerase a sliding grip on the minor groove of the replicated DNA, dramatically increasing processivity without compromising catalytic activity or enzyme stability. This enzyme, when combined with a special buffer formulation containing the EvaGreen dye, becomes a powerful research tool that provides not only high tolerance to PCR inhibitors but also short reaction times and the ability to synthesize long PCR products.

Here we describe a novel real-time PCR assay for the detection of mycoplasma contamination in tissue culture samples using a single copy gene as a DNA target. We have designed primers targeting conserved sequences in the rpoB gene which allow the specific detection of 8 common mycoplasma species. Our data demonstrate that the rpoB PCR based on the SsoFast EvaGreen Supermix allows the detection of mycoplasma in tissue culture supernatants with minimal or no interference by PCR inhibitors. The assay is resistant to various common media formulations and FBS at 10% final concentration. No inhibition was found by conditioned media from cells. The sensitivity of our assay is between 1 and 6 gene copies. The assay is specific to mycoplasma; no crossreactivity was observed when testing other common tissue culture contaminations. Finally we validated our real-time PCR assay for reliable detection of mycoplasma in crude tissue culture samples using mycoalert kit from Lonza as a standard.

### Materials and Methods

Genomic DNA from the following species was purchased from ATCC: A. laidlawii (23206D); M. orale (23714D); M. arginini (23838D); M. fermentans (19989D); M. hominis, strain PG21 (23114D); M. pirum (25960D); M. hyorhinis (17981D); Candida albicans (10231D-5); lactobacillus casei (334D); Bacillus subtilis (23857D); Genomic DNA of M. salivarium (NCTC010113) was purchased from Minerva BioLabs. Iscoves media was purchased from ATCC, FBS from Hyclone, EGM2 from Lonza, and Esgro complete from Chemicon, L15 from Sigma. RPMI, Neurobasal media, DMEM, DMEM/F12, F12K, KSFM, OPTIMEM, MEM and MEM ALPHA were from Gibco. All media (except KSFM, Neurobasal, ESGRO complete, OPTIMEM) were supplemented with 10% FBS. Contaminated and non-infected tissue culture cell lines were obtained from various collaborators. Primers were obtained from IDT. The sequence of the primers are: forward primer 5′-GAAGAWATGCCWTATTTAGAAGATGG-3′; reverse primer 5′-CCRTTTTGACTYTTWCCACCMAGTGGTTGTTG-3′. Real-time PCR reactions were performed using the SsoFast EvaGreen Supermix (Bio-Rad), the indicated templates, and 0.5 micromolar final primer concentration in a volume of 20 microliter on the Bio-Rad CFX96 real-time PCR detection system with the following conditions: initial denaturation: 94° C., 3 min., and 40 cycles of 94° C., 10 sec., 58° C., 30 sec., 72° C., 30 sec. Data were analyzed using the CFX data manager software.

Mycoalert mycoplasma detection kit was obtained from Lonza and used according to the manufacturer's recommendations. Luminescence readings were performed on Lumimark from Bio-Rad.

### Results

Design of Universal rpoB Primers for Real-Time PCR

The rpoB gene has been previously described for the detection of mycoplasma contamination in cell culture by conventional PCR (Kong, H. et al. (2007) Appl Microbiol Biotechnol, 77, 223-232). To design primers suitable for real-time PCR, the sequences of eight mycoplasma species (M. pirum, A. laidlawii; M. arginini; M. orale; M. salivarium; M. hominis; M. fermentans; M. hyorhinis.) were aligned using software developed by Accelrys. Two conserved regions spanning 400-600 bp were chosen as primer binding sites and mixed bases were used at various less conserved positions in order to detect the eight most common species found in cell culture contaminations (FIG. 1).

To confirm that the primers are able to detect all eight species, PCR reactions were conducted with eight common mycoplasma species. Realtime-PCR were set up using 20 pg of genomic DNA, the rpoB primers and SsoFast EvaGreen Supermix. After the PCR reaction, DNA fragments were analyzed by agarose gel electrophoresis. As shown in FIG. 2A, the rpoB primers allowed the detection of all tested mycoplasma species between cycle 22 and 27. The melt peak temperature was between 77.5 and 80.5 degrees. The size of the PCR products was 600 bp for M. pirum and 400 bp for all other species (FIG. 2B), as predicted from the sequence alignment. In summary these data indicate that the rpoB primers are suitable for the specific detection of the most common mycoplasma.

We tested the cross reactivity of the primers against a variety of other species. Because tissue culture cells can release DNA into the media, we tested human, mouse, and rat genomic DNA, as these are common sources of tissue culture cells. We also tested phylogenetically related bacteria (Lactobacillus casei, Bacillus subtilis) and common non mycoplasma tissue culture contaminants (E. coli, Candida albicans). One ng of genomic DNA from the various species was subjected to PCR using the rpoB primers along with the EvaGreen supermix. As shown in FIG. 2C, only genomic DNA from Lactobacillus casei resulted in PCR amplification. The detection limit for Lactobacillus casei genomic DNA was about 10 pg (data not shown). This corresponds to about 3×10⁶ bacteria/ml, which would be apparent as classical bacterial contamination by other means, e.g., microscopical inspection. Therefore it is very unlikely that Lactobacillus casei contamination could be mistaken as mycoplasma contamination. The other DNA templates did not give rise to specific signal. These data indicate that the rpoB primers are suitable for the specific detection of the eight most common mycoplasma species found in contaminated cell cultures.

### Sensitivity of the PCR

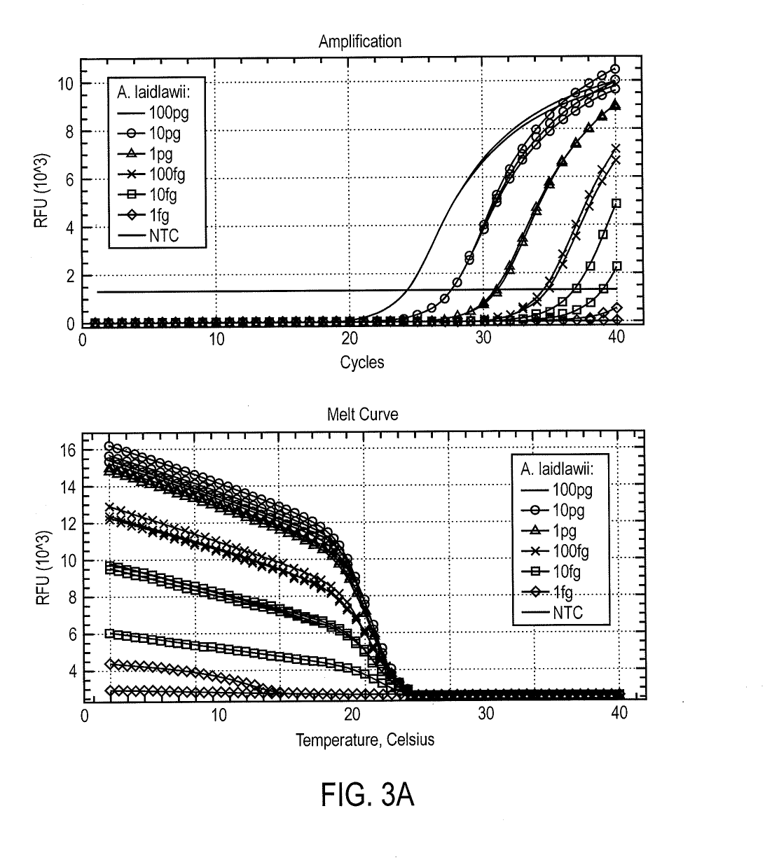

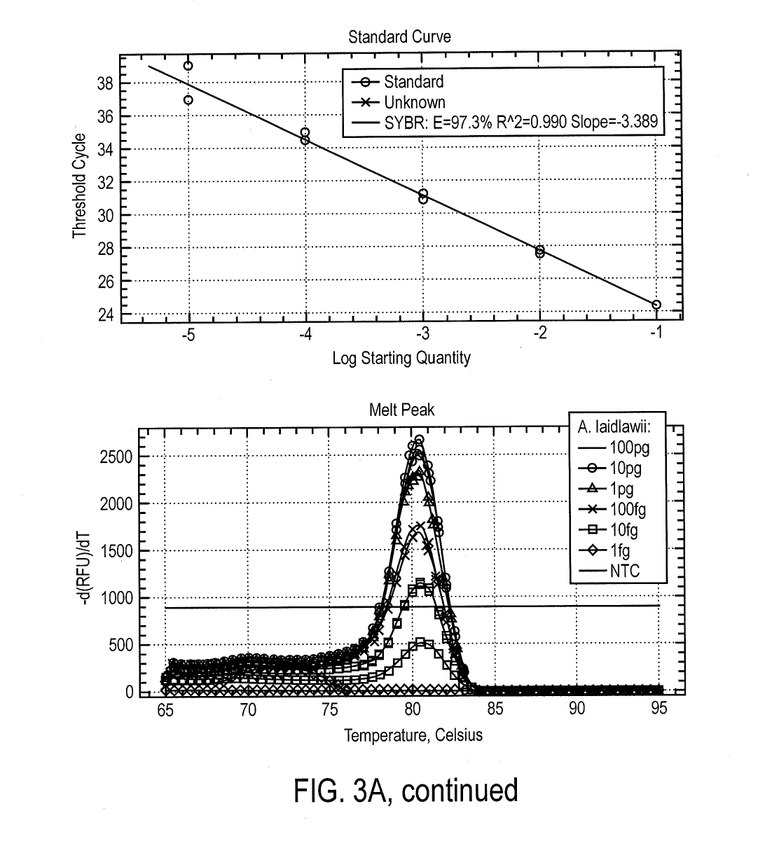

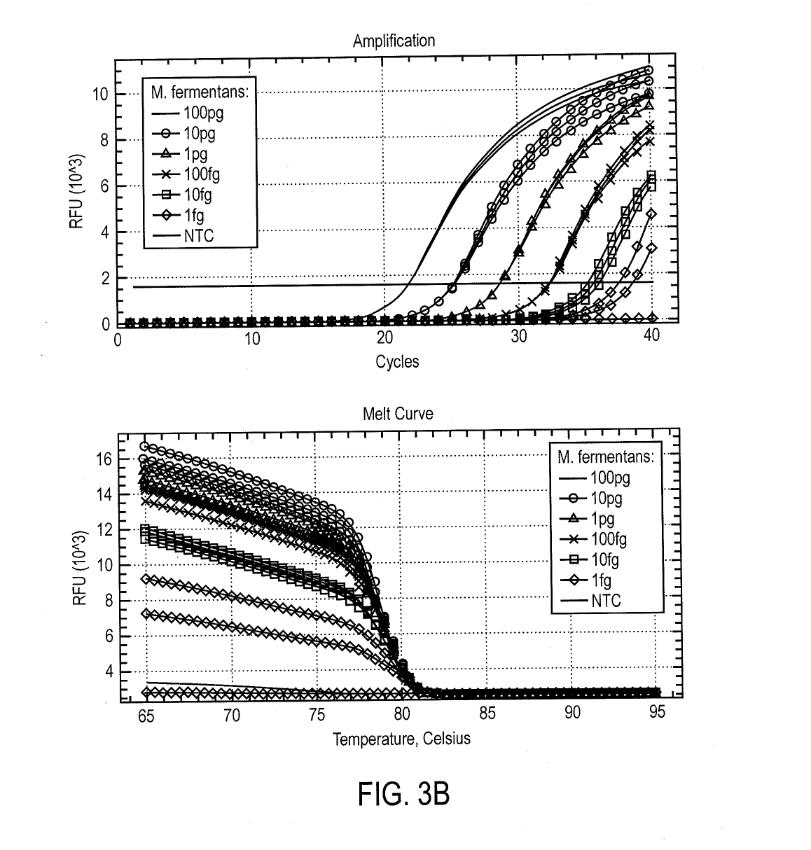

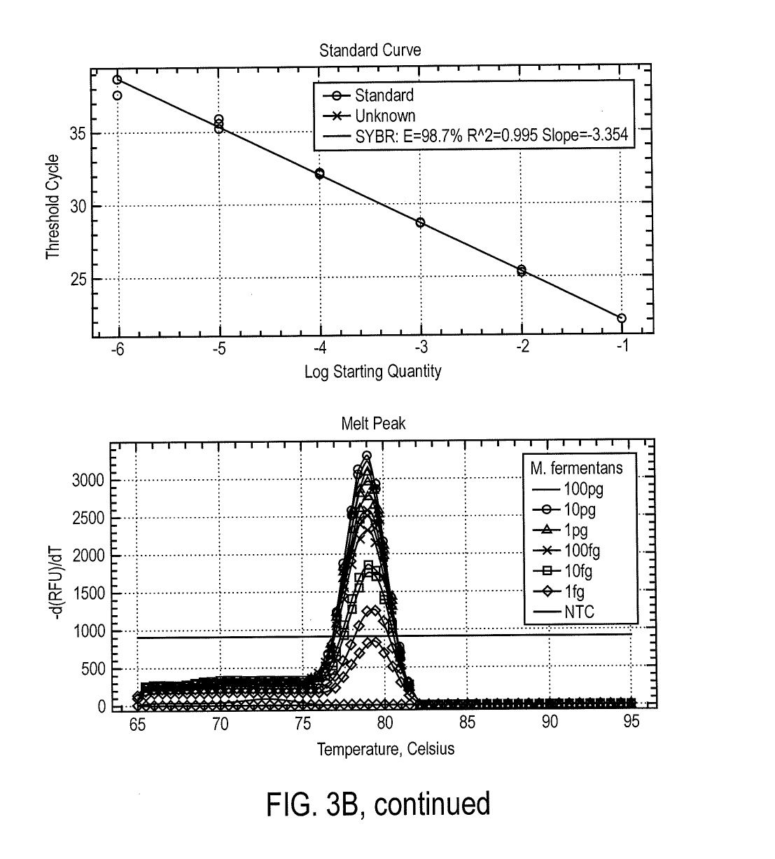

Because of sequence differences in the rpoB gene among the mycoplasma species, the sensitivity of the PCR assay can lead to different detection limits among the species. To investigate the sensitivity of the assay, we determined the limit of detection for two representative mycoplasma species. A. laidlawii was chosen because the sequence of its rpoB gene is less conserved and exhibits mismatches with our primers (FIG. 1B, labeled L) and resulted in later Ct values than the other species (FIG. 2A, line with circle). The DNA sequence of the rpoB gene from M. fermentans, is almost identical with the primer sequences (FIG. 1B, labeled F) and was detected with an early Ct in our method (FIG. 2A, line with cross). To determine the range over which the assay is successful for these two species, ten-fold serial dilutions of genomic DNA, from 100 pg-1 fg, were subjected to the rpoB PCR assay. 10 fg was the detection limit for the less conserved species A. laidlawii (FIG. 3A), which is equivalent to about 6 copies of the genome, given a genome size of 1497 kb (NC_{—}010163.1). When using genomic DNA from M. fermentans as template (FIG. 3B), 1 fg of genomic DNA was detected which is equivalent to approximately one copy, based on a genome size of 1245 bp (Schaeverbeke, T. et al. (1998) J Clin Microbiol, 36, 1226-1231). Estimation of very low copy numbers is best done through a digital PCR like approach where the number of successful amplifications among a large number of replicates is used to determine copy number rather than the Ct (Sykes, P. J. et al. (1992) Biotechniques, 13, 444-449); assuming a Poisson distribution of template molecules, only 66% of single copy reactions should contain a target and therefore amplify, and corresponding fewer with less than 1 copy (Sykes, P. J. et al. (1992) Biotechniques, 13, 444-449). To confirm single copy detection, we amplified 75 replicate samples with 1 fg M. fermentans genomic DNA as template. The rpoB amplicon was successfully amplified in 53% of the reactions which matches the expectation based on 0.8 genomic equivalents (data not shown). Therefore, our method is highly sensitive and capable of detecting approximately one to six genomic copies per PCR reaction.

### Resistance to PCR Inhibitors

One of the useful properties of the SsoFast EvaGreen Supermix is its resistance to PCR inhibitors present in crude samples such as blood or serum. We tested the performance of the SsoFast EvaGreen Supermix in the presence of various cell culture media, including FBS. The media were included into the real-time PCR reactions at 10% final concentration. To test the performance characteristics of the mix, we used purified genomic DNA derived from mycoplasma infected samples as template for the PCR. As shown in Table 1, the amplification of purified DNA alone resulted in an average Ct of 27. When various media formulations were added to the PCR reactions, we observed a small Ct delay (less than 0.5 Ct). Only FBS showed a delay of almost 1 Ct. These data indicate that the SsoFast EvaGreen Supermix generates highly efficient PCR amplifications in the presence of various tissue culture media and also in the presence of 10% FBS (final concentration).

TABLE 1

| | Average | STDEV |
|---|---|---|
| Sample | C(t) | C(t) |
| water | 27.00 | 0.11 |
| RPMI | 27.30 | 0.04 |
| Optimem | 27.13 | 0.06 |
| Neurobasal | 27.05 | 0.03 |
| MEM | 27.33 | 0.11 |
| Iscoves | 27.45 | 0.05 |
| FBS | 27.97 | 0.06 |
| F12K | 27.24 | 0.06 |
| ESGRO | 27.26 | 0.03 |
| EGM2 | 27.38 | 0.03 |
| DMEM | 27.48 | 0.05 |

It has been suggested that not only media components but also cell debris and excessive amounts of DNA (derived from apoptotic cells) can result in PCR inhibition (Kong, H. et al. (2007) Appl Microbiol Biotechnol, 77, 223-232; Harasawa, R. et al. (2005) Microbiol Immunol, 49, 859-863). To test this hypothesis we examined whether conditioned media from aged, over confluent cell cultures would inhibit PCR. As shown in Table 2, the addition of various types of conditioned media caused only a minor Ct delay (less than 1 Ct), thus indicating that PCR reactions performed with the SsoFast EvaGreen Supermix are minimally affected by the presence of PCR inhibitors.

TABLE 2

| | Average | STDEV |
|---|---|---|
| sample | C(t) | C(t) |
| water | 26.66 | 0.03 |
| RPMI, conditioned | 26.88 | 0.04 |
| Neurobasal, conditioned | 26.94 | 0.03 |
| MEM, conditioned | 27.03 | 0.05 |
| EGM2, conditioned | 26.88 | 0.06 |
| DMEM, conditioned | 27.07 | 0.08 |

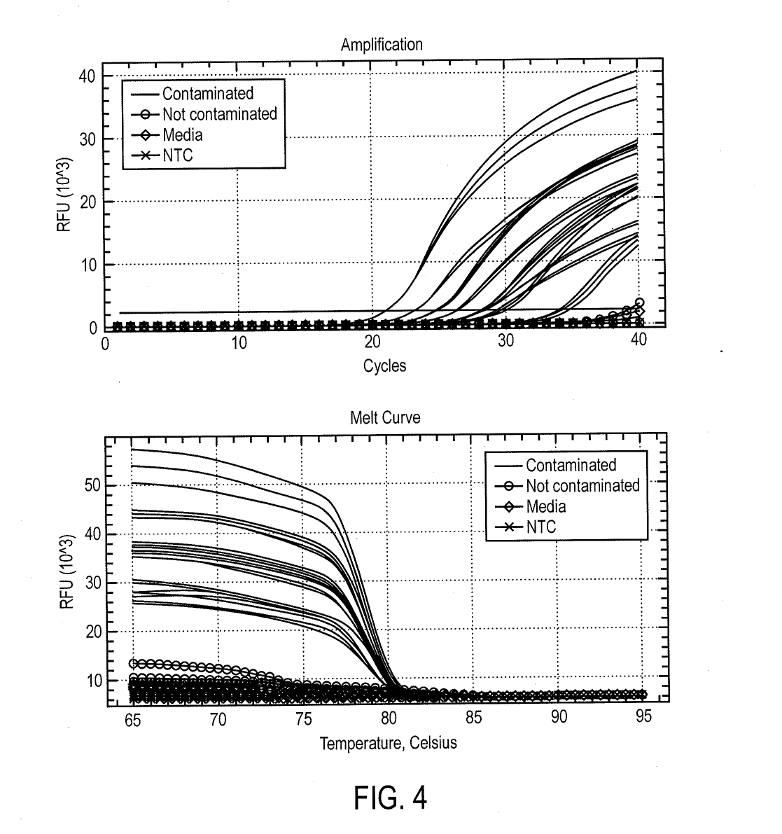

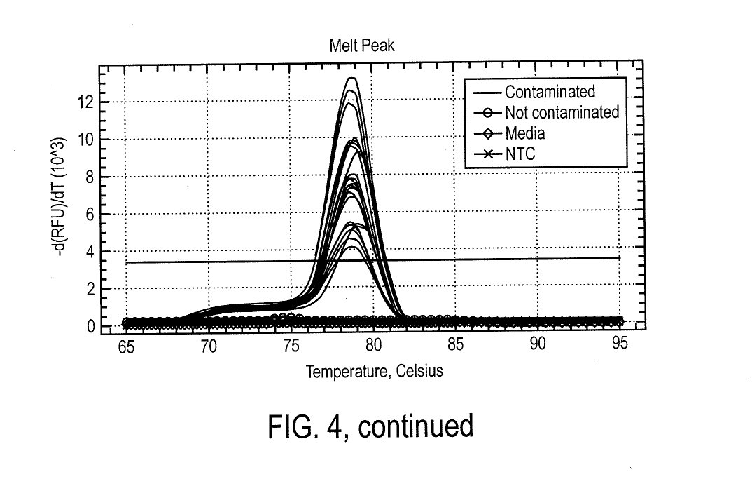

Finally, we tested the performance of our real-time PCR assay for mycoplasma detection in crude samples without prior sample preparation. Using the commercially available mycoplasma detection kit from Lonza, we screened a variety of cell culture supernatant samples and identified 10 contaminated and 10 non contaminated samples (data not shown). We tested those samples along with a media control and a non-template control in our real-time PCR assay. The cell culture supernatants were directly subjected to PCR without any sample preparation. We were able to detect mycoplasmas in all 10 infected samples (plain line, FIG. 4A), while the non infected samples did not result in specific amplification (line with circles). Ct values between 21 and 34 and melt peaks between 78.5 and 79° C. were obtained for the positive samples (Table 3), while the non infected samples and the control samples generated Cts larger than 39 (with no specific melt peak present).

TABLE 3

| | | average | STDEV | average | |
|---|---|---|---|---|---|
| cell type | species | C(t) | C(t) | melt temp | result |
| Xenopus S3 | Xenopus | | | | negative |
| C6 | human | | | 74.00 | negative |
| CHO | Chinese | | | | negative |
| | hamster | | | | |
| Hela S3 | human | | | | negative |
| 293T | human | | | | negative |
| Hela | human | | | | negative |
| MC3T3 | mouse | | | | negative |
| A549 | human | | | | negative |
| Jurkat | human | | | | negative |
| HUVEC | human | 39.36 | | 74.50 | negative |
| Phoenix | human | 26.36 | 0.03 | 79.00 | positive |
| 293T | human | 26.46 | 0.08 | 79.00 | positive |
| PC3 | human | 25.10 | 0.09 | 78.50 | positive |
| VCaP | human | 34.56 | 0.46 | 78.50 | positive |
| PC3 | human | 23.33 | 0.05 | 79.00 | positive |
| RWPE-1 | human | 21.17 | 0.04 | 78.67 | positive |
| A549 | human | 28.77 | 0.27 | 79.00 | positive |
| CH3/10T1/2 | mouse | 30.29 | 0.13 | 78.50 | positive |
| PAC1 | rat | 29.63 | 0.07 | 78.50 | positive |
| MDA-MB-231 | human | 28.49 | 0.14 | 78.67 | positive |

### Discussion

Mycoplasmas are a serious problem in tissue culture that can lead to erroneous results. Unlike typical bacterial or fungal contaminants which can be easily seen, mycoplasma contamination is not readily detectable without specific testing so it can easily go undetected. Therefore regular testing is highly recommended, but not always performed, due to the issues with current testing methods. We present here a novel real-time PCR assay for the detection of mycoplasma in cell culture supernatants. The major advantages of our protocol are improved specificity and sensitivity, avoiding extensive sample preparation, HT sample throughput, simple reaction set up, and speed. These advantages make the testing for mycoplasma contamination much more practical.

Several protocols have been described in the literature for real-time PCR detection of mycoplasma, but most of them rely on the detection of ribosomal DNA sequences (Ishikawa, Y. et al., (2006) In Vitro Cell Dev Biol Anim, 42, 63-69; Harasawa, R. et al. (2005) Microbiol Immunol, 49, 859-863; Schmitt, M. and Pawlita, M. (2009) Nucleic Acids Res.). A major disadvantage of those genes is that due to the high conservation these primers are not specific to mycoplasma but may also detect other phylogenetically related bacteria (Tang, J. et al., (2000) J Microbiol Methods, 39, 121-126; Eldering, J. A. et al. (2004) Biologicals, 32, 183-193). It is possible to improve the specificity of the PCR reactions by incorporating specific probes that do not detect the non-target species (Schmitt, M. and Pawlita, M. (2009) Nucleic Acids Res.; Promokine), But this approach results in higher cost of material.

To avoid species cross-reactivity, other less conserved mycoplasma genes have been used for PCR detection. Stormer et al. developed an assay for the amplification of the tuf gene using broad range primers and a mycoplasma-specific probe (Stormer, M. et al. (2009) Int J Med Microbiol, 299, 291-300). Their assay shows no cross-reactivity with 33 bacterial and fungal species, but no data are available regarding the detection of the mycoplasma species M. arginini and M. pirum (Stormer, M. et al. (2009) Int J Med Microbiol, 299, 291-300), two common cell culture contaminants (Tang, J. et al., (2000) J Microbiol Methods, 39, 121-126; Timenetsky, J. et al. (2006) Braz J Med Biol Res, 39, 907-914). In our hands, the tuf primers failed to detect these two species (data not shown). We believe that the assay presented by Stormer is highly specific to mycoplasma and well optimized for the detection of clinically relevant mollicute species, but not sufficient for testing of all common mycoplasma species in cell culture.

As an alternative approach, we designed primers suitable for real-time PCR, targeting a region of the rpoB gene, that allow the specific detection of at least eight common species of mycoplasma (FIG. 2A, B). We observed amplification from genomic DNA of all eight of the mycoplasma spp. tested and only one related non-target bacterium, Lactobacillus casei, suggesting these primers already combine good specificity with broad coverage among mycoplasma spp. (FIG. 2).

The sensitivity of the rpoB assay was determined to be approximately 1 to 6 genome copies per reaction, depending on the species detected (FIG. 3). Typical mycoplasma infected cultures contain between 10⁵-10⁸ colony forming units per ml (Wirth, M. et al. (1994) Cytotechnology, 16, 67-77). Therefore this method is sensitive enough to detect even weakly infected cultures.

When testing equal genomic DNA amounts of the eight most common species, we observed a difference of 5 Cts between the earliest and latest detected species. This may be explained by the fact that the rpoB sequences chosen for primer binding sites of the eight most common species are not entirely homologous, resulting in differences in primer binding and PCR amplification efficiencies among the different targets. Another possible explanation is differences in quality and quantity of the starting material of genomic DNA obtained from two different vendors.

We show here that our method does not require sample preparation prior to detection. We tested the most common media formulations including FBS as well as conditioned media containing cell debris and cellular waste products. We found that none of the tested media led to more than a 1 Ct delay, indicating that our PCR assay is highly resistant to PCR inhibitors (Table 1-2). The elimination of the sample preparation step provides several advantages: DNA purification is a labor intensive procedure; since this is no longer necessary for our method, many samples can be processed simultaneously. Elimination of the sample preparation step also improves the quality of the assay, since there are fewer chances for sample loss and error. Our method is also appealing since it is rapid and the reactions are easy to set up. Results can be obtained in less than 2 hours with very little handling time so testing is no longer a large drain on laboratory resources.

To test the effectiveness of the rpoB assay in real samples, we investigated a set of 10 infected and 10 non-infected cultures that were identified by a commercially available biochemical mycoplasma test kit (Mariotti, E. et al. (2008) Leuk Res, 32, 323-326). The rpoB assay obtained the same results on all 20 cell culture samples as the commercially available kit (FIG. 4, Table 3), suggesting that our method is suitable for regular testing of mycoplasma infected samples. Our results differed from the biochemically based kit only with regard to one control sample as the kit identified a sample of uninoculated media as positive while our rpoB assay did not (data not shown). One known disadvantage of the biochemical method is the occurrence of false positives (Sykes, P. J. et al. (1992) Biotechniques, 13, 444-449). This may be due to the fact that the test relies on the detection of ATP, which is fairly abundant in the lab environment.

Taken together we show here a simple mycoplasma detection method that is suitable for regular testing in laboratories to prevent spreading of mycoplasma infections.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### INFORMAL SEQUENCE LISTING

SEQ ID NO: 1
GAAGAWATGCCWTATTTAGAAGATGG
SEQ ID NO: 2
CCRTTTTGACTYTTWCCACCMAGTGGTTGTTG
SEQ ID NO: 3
ATGATCCTGGATGCTGACTACATCACTGAAGAAGGCAAACCGGTTATC
CGTCTGTTCAAAAAAGAGAACGGCGAATTTAAGATTGAGCATGATCGC
ACCTTTCGTCCATACATTTACGCTCTGCTGAAAGATGATTCTAAGATT
GAGGAAGTTAAAAAAATCACTGCTGAGCGCCATGGCAAGATTGTTCGT
ATCGTTGATGCGGAAAAGGTAGAAAAGAAATTTCTGGGCAGACCAATC
ACCGTGTGGAGACTGTATTTCGAACATCCACAAGATGTTCCGACTATT
CGCGAGAAAATTCGCGAACATTCTGCAGTTGTTGACATCTTCGAATAC
GATATTCCATTTGCAAAGCGTTACCTCATCGACAAAGGCCTGATACCA
ATGGAGGGCGATGAAGAACTCAAGCTCCTGGCGTTCGCTATAGCAACC
CTCTATCACGAAGGCGAAGAGTTTGGTAAAGGCCCAATTATAATGATC
AGCTATGCAGATGAAGAAGAAGCAAAGGTGATTACTTGGAAAAAAATA
GATCTCCCATACGTTGAGGTTGTATCTTCCGAGCGCGAGATGATTAAG
CGCTTTCTCAAAATTATCCGCGAGAAGGATCCGGACATTATCATTACT
TATAACGGCGACTCTTTTGACCTCCCATATCTGGCGAAACGCGCAGAA
AAACTCGGTATTAAACTGACTATCGGCCGTGATGGTTCCGAGCCGAAG
ATGCAGCGTATCGGCGATATGACCGCTGTAGAAGTTAAGGGTCGTATC
CATTTCGACCTGTATCATGTAATTCGTCGTACTATTAACCTCCCGACT
TACACTCTCGAGGCTGTATATGAAGCAATTTTTGGTAAGCCGAAGGAG
AAGGTATACGCCGATGAGATTGCAAAGGCGTGGGAAACCGGTGAGGGC
CTCGAGCGTGTTGCAAAATACTCCATGGAAGATGCAAAGGCGACTTAT
GAACTCGGCAAAGAATTCTTCCCAATGGAAGCTCAGCTCTCTCGCCTG
GTTGGCCAACCACTGTGGGATGTTTCTCGTTCTTCCACCGGTAACCTC
GTAGAGTGGTTTCTCCTGCGCAAAGCGTACGAACGCAACGAACTGGCT
CCGAACAAGCCAGATGAACGTGAGTATGAACGCCGTCTCCGCGAGTCT
TACGCTGGTGGCTTTGTTAAAGAGCCAGAAAAGGGCCTCTGGGAAAAC
ATCGTGTCCCTCGATTTTCGCGCTCTGTATCCGTCTATTATCATTACC
CACAACGTGTCTCCGGATACTCTCAACCGCGAGGGCTGCAGAAACTAT
GATGTTGCTCCGGAAGTAGGCCACAAGTTCTGCAAGGACTTCCCGGGC
TTTATTCCGTCTCTCCTGAAACGTCTGCTCGATGAACGCCAAAAGATT
AAGACTAAAATGAAGGCGTCCCAGGATCCGATTGAAAAAATAATGCTC
GACTATCGCCAAAGAGCGATTAAAATCCTCGCAAACTCTTATTACGGC
TATTATGGCTATGCAAAAGCACGCTGGTACTGTAAGGAGTGTGCTGAG
TCCGTTACTGCTTGGGGTCGCGAATACATCGAGTTCGTGTGGAAGGAG
CTCGAAGAAAAGTTTGGCTTTAAAGTTCTCTACATTGACACTGATGGT
CTCTATGCGACTATTCCGGGTGGTAAGTCTGAGGAAATTAAGAAAAAG
GCTCTAGAATTTGTGGATTACATTAACGCGAAGCTCCCGGGTCTCCTG
GAGCTCGAATATGAAGGCTTTTATAAACGCGGCTTCTTCGTTACCAAG
AAGAAATATGCGCTGATTGATGAAGAAGGCAAAATTATTACTCGTGGT
CTCGAGATTGTGCGCCGTGATTGGAGCGAAATTGCGAAAGAAACTCAA
GCTAGAGTTCTCGAGGCTATTCTCAAACACGGCAACGTTGAAGAAGCT
GTGAGAATTGTAAAAGAAGTAACCCAAAAGCTCTCTAAATATGAAATT
CCGCCAGAGAAGCTCGCGATTTATGAGCAGATTACTCGCCCGCTGCAT
GAGTATAAGGCGATTGGTCCGCACGTGGCTGTTGCAAAGAGACTGGCT
GCTAAAGGCGTGAAAATTAAACCGGGTATGGTAATTGGCTACATTGTA
CTCCGCGGCGATGGTCCGATTAGCAACCGTGCAATTCTAGCTGAGGAA
TACGATCCGAGAAAGCACAAGTATGACGCAGAATATTACATTGAGAAC
CAGGTGCTCCCGGCGGTACTCCGTATTCTGGAGGGTTTTGGCTACCGT
AAGGAAGACCTCCGCTGGCAAAAGACTAAACAGACTGGCCTCACTTCT
TGGCTCAACATTAAAAAATCCGGTACCGGCGGTGGCGGTGCAACCGTA
AAGTTCAAGTACAAAGGCGAAGAAAAAGAGGTAGACATCTCCAAGATC
AAGAAAGTATGGCGTGTGGGCAAGATGATCTCCTTCACCTACGACGAG
GGCGGTGGCAAGACCGGCCGTGGTGCGGTAAGCGAAAAGGACGCGCCG
AAGGAGCTGCTGCAGATGCTGGAGAAGCAGAAAAAGTGA
SEQ ID NO: 4
MILDADYITEEGKPVIRLFKKENGEFKIEHDRTFRPYIYALLKDDSKI
EEVKKITAERHGKIVRIVDAEKVEKKFLGRPITVWRLYFEHPQDVPTI
REKIREHSAVVDIFEYDIPFAKRYLIDKGLIPMEGDEELKLLAFAIAT
LYHEGEEFGKGPIIMISYADEEEAKVITWKKIDLPYVEVVSSEREMIK
RFLKIIREKDPDIIITYNGDSFDLPYLAKRAEKLGIKLTIGRDGSEPK
MQRIGDMTAVEVKGRIHFDLYHVIRRTINLPTYTLEAVYEAIFGKPKE
KVYADEIAKAWETGEGLERVAKYSMEDAKATYELGKEFFPMEAQLSRL
VGQPLWDVSRSSTGNLVEWFLLRKAYERNELAPNKPDEREYERRLRES
YAGGFVKEPEKGLWENIVSLDFRALYPSIIITHNVSPDTLNREGCRNY
DVAPEVGHKFCKDFPGFIPSLLKRLLDERQKIKTKMKASQDPIEKIML
DYRQRAIKILANSYYGYYGYAKARWYCKECAESVTAWGREYIEFVWKE
LEEKFGFKVLYIDTDGLYATIPGGKSEEIKKKALEFVDYINAKLPGLL
ELEYEGFYKRGFFVTKKKYALIDEEGKIITRGLEIVRRDWSEIAKETQ
ARVLEAILKHGNVEEAVRIVKEVTQKLSKYEIPPEKLAIYEQITRPLH
EYKAIGPHVAVAKRLAAKGVKIKPGMVIGYIVLRGDGPISNRAILAEE
YDPRKHKYDAEYYIENQVLPAVLRILEGFGYRKEDLRWQKTKQTGLTS
WLNIKKSGTGGGGATVKFKYKGEEKEVDISKIKKVWRVGKMISFTYDE
GGGKTGRGAVSEKDAPKELLQMLEKQKK*
SEQ ID NO: 5
TGC ATT TTG TCA TCA ACC ATG TG
SEQ ID NO: 6
CCT TCA CGT ATG AAC AT
M pirum
Forw
SEQ ID NO: 7
GAAGATATGCCGCATTTAGAAGATGGC
M pirum
Rev
SEQ ID NO: 8
CAACAACCATTGGGTGGTAAATCACAAAATGGT
A. laidlawii
Forw
SEQ ID NO: 9
GAAGATATGCCATATTTAGCAGATGGA
A. laidlawii
Rev
SEQ ID NO: 10
CAACAACCAATGGGTGGTAAAGCTCAAAACGGT
M. arginini
Forw
SEQ ID NO: 11
GAAGAAATGCCATACTTAGAAGATGGA
M. arginini
Rev
SEQ ID NO: 12
CAACAACCACTTGGTGGTAAATCACAAAATGGT
M. orale
Forw
SEQ ID NO: 13
GAAGAAATGCCTTATTTAGAAGATGGT
M. orale
Rev
SEQ ID NO: 14
CAACAACCTCTTGGTGGTAAAAGTCAAAACGGT
M. salivarium
Forw
SEQ ID NO: 15
GAAGATATGCCTTATTTAGAAGATGGA
M. salivarium
Rev
SEQ ID NO: 16
CAACAACCACTTGGTGGAAAGAGTCAAAATGGT
M. hominis
Forw
SEQ ID NO: 17
GAAGAAATGCCTTATTTAGCAGATGGA
M. hominis
Rev
SEQ ID NO: 18
CAACAACCACTTGGTGGAAAGAGTCAAAATGGT
M. fermentans
Forw
SEQ ID NO: 19
GAAGATATGCCTTATTTAGAAGATGGT
M. fermentans
Rev
SEQ ID NO: 20
CAACAACCTCTTGGAGGTAAGAGTCAAAACGGT
M. hyorhinis
Forw
SEQ ID NO: 21
GAAGATATGCCATTTTTAGAAGATGGA
M. hyorhinis
Rev
SEQ ID NO: 22
CAACAACCACTTGGAGGAAAAAGTCAAAACGGT
FIG. 1 Forward consensus
SEQ ID NO: 23
GAAGAWATGCCWTATTTAGAAGATGG
FIG. 1 Reverse consensus
SEQ ID NO: 24
CAACAACCACTKGGTGGWAARAGTCAAAAYGG

What is claimed is:

1. A method of detecting mycoplasma in a cell culture media, the method comprising,
obtaining an aliquot of cell culture media;
performing a real-time nucleic acid amplification reaction with a thermostable DNA polymerase to amplify a mycoplasma nucleic acid, if present, in the aliquot wherein:
nucleic acids in the aliquot are not further purified, and
the amplification reaction comprises an intercalating fluorescent dye that produces a fluorescent signal in the presence of double stranded DNA; and
detecting a melting temperature of an amplification product of the amplification reaction, wherein the presence of an amplification product indicates the presence of mycoplasma in the cell culture.

2. The method of claim 1, wherein the amplification reaction is capable of amplifying any of Mycoplasma arginini, M. pirum, M. hominis, M. fermentans, M. salivarium, M. orale, M. hyorhinis and Acholeplasma laidlawii, if present in the aliquot.

3. The method of claim 1, wherein the mycoplasma nucleic acid comprises a portion of at least 50 nucleotides of a rpoB gene.

4. The method of claim 1, wherein the performing step comprises amplifying the portion with the following degenerate primers:

| | |
|---|---|
| GAAGAWATGCCWTATTTAGAAGATGG; | (SEQ ID NO: 1) |
| and | |
| CCRTTTTGACTYTTWCCACCMAGTGGTTGTTG, | (SEQ ID NO: 2) |

wherein W represents A or T, Y represents C or T, R represents A or G, and M represents A or C.

5. The method of claim 4, wherein the reaction contains no more primers than one forward primer and one reverse primer.

6. The method of claim 1, wherein the detecting step further comprises nucleotide sequencing the amplification product and correlating the determined nucleotide sequence to nucleotide sequences of different mycoplasma species, thereby determining the identity of the mycoplasma.

7. The method of claim 1, wherein the amplification reaction does not comprise a detectably-labeled oligonucleotide.

8. The method of claim 1, wherein the polymerase is linked to a sequence non-specific DNA binding domain.

9. The method of claim 8, wherein the sequence non-specific DNA binding domain is an Sso7 DNA binding domain.

10. The method of claim 1, wherein the aliquot comprises a sufficient amount of an amplification inhibitor to inhibit activity of Taq polymerase by at least 10%.

11. The method of claim 10, wherein the inhibitor is selected from the group consisting of cell debris, cell waste products (e.g., polysaccharides or proteins), and fetal bovine serum or an amplification inhibitor component thereof.

12. The method of claim 1, wherein the amplification reaction comprises a sufficient amount of an osmolyte and/or heparin to improve efficiency of the amplification reaction.

13. The method of claim 12, wherein the osmolyte is selected from the group consisting of sarcosine, trimethylamine N-oxide (TMAO), dimethylsulfoniopropionate, and trimethylglycine.

14. A method of detecting mycoplasma in a cell culture media, the method comprising,
obtaining an aliquot of cell culture media;
performing a nucleic acid amplification reaction with a DNA polymerase to amplify a mycoplasma nucleic acid, if present, in the aliquot wherein:
(a) the performing step comprises amplifying the portion with a first and second primer, the first primer comprising the following degenerate sequence:
GAAGAWATGCCWTATTTAGAAGATGG (SEQ ID NO:1); and
the second primer comprising the following degenerate sequence:
CCRTTTTGACTYTTWCCACCMAGTGGTTGTTG (SEQ ID NO:2), wherein W represents A or T, Y represents C or T, R represents A or G, and M represents A or C; and
(b) detecting the presence or absence of an amplification product of the amplification reaction, wherein the presence of an amplification product indicates the presence of mycoplasma in the cell culture.

15. The method of claim 14, wherein the amplification reaction is monitored in real-time.

16. The method of claim 15, wherein the amplification reaction comprises an intercalating fluorescent dye that produces a fluorescent signal in the presence of double stranded DNA at least twice that produced in the presence of single-stranded DNA only.

17. The method of claim 14, wherein the first and second primers consist of the following degenerate sequences, respectively:

| | |
|---|---|
| GAAGAWATGCCWTATTTAGAAGATGG; | (SEQ ID NO: 1) |
| and | |
| CCRTTTTGACTYTTWCCACCMAGTGGTTGTTG | (SEQ ID NO: 2) |

18. The method of claim 14, wherein nucleic acids in the aliquot are not further purified.

19. The method of claim 14, wherein the reaction contains no more than one forward and one reverse primer designed to hybridize to an rpoB gene.

20. The method of claim 14, wherein the detecting step further comprises nucleotide sequencing the amplification product and correlating the determined nucleotide sequence to nucleotide sequences of different mycoplasma species, thereby determining the identity of the mycoplasma.

21. The method of claim 14, wherein the amplification reaction does not comprise a detectably-labeled oligonucleotide.

22. The method of claim 14, wherein the polymerase is linked to a sequence non-specific DNA binding domain.

23. The method of claim 22, wherein the sequence non-specific DNA binding domain is an Sso7 DNA binding domain.

24. The method of claim 14, wherein the amplification reaction comprises a sufficient amount of an osmolyte and/or heparin to improve efficiency of the amplification reaction.

25. The method of claim 24, wherein the osmolyte is selected from the group consisting of sarcosine, trimethylamine N-oxide (TMAO), dimethylsulfoniopropionate, and trimethylglycine.

26. A kit for amplifying mycoplasma DNA, if present, from cell culture media, the kit comprising:
a first degenerate primer comprising GAAGAWATGCCWTATTTAGAAGATGG (SEQ ID NO:1); and
a second degenerate primer comprising CCRTTTTGACTYTTWCCACCMAGTGGTTGTTG (SEQ ID NO:2),
wherein W represents A or T, Y represents C or T, R represents A or G, and M represents A or C.

27. The kit of claim 26, wherein the first primer consists of GAAGAWATGCCWTATTTAGAAGATGG (SEQ ID NO:1); and
the second primer consists of CCRTTTTGACTYTTWCCACCMAGTGGTTGTTG (SEQ ID NO:2).

28. The kit of claim 26, further comprising at least one or more of the following:
a polymerase;
an intercalating fluorescent dye;
a positive control polynucleotide comprising a polynucleotide that can be amplified by a polymerase primed by SEQ ID NO:1 or SEQ ID NO:2.

29. The kit of any of claims 26-28, wherein the kit further comprises a positive control sample comprising a nucleic acid comprising at least 50 contiguous nucleotides of a Mycoplasma rpoB gene.

30. The kit of claim 26, further comprising an osmolyte and/or heparin.

31. The kit of claim 30, wherein the osmolyte is selected from the group consisting of sarcosine, trimethylamine N-oxide (TMAO), dimethylsulfoniopropionate, and trimethylglycine.

32. The kit of claim 28, wherein the polymerase is linked to a sequence non-specific DNA binding domain.

33. The kit of claim 32, wherein the sequence non-specific DNA binding domain is an Sso7 DNA binding domain.


SEQUENCE LISTING (27 sequences)

SEQ ID NO: 1 - DNA, 26 bp, Artificial Sequence
  synthetic rpoB gene first degenerate primer
gaagawatgccwtatttagaagatgg

SEQ ID NO: 2 - DNA, 32 bp, Artificial Sequence
  synthetic rpoB gene second degenerate primer
ccrttttgactyttwccaccmagtggttgttg

SEQ ID NO: 3 - DNA, 2535 bp, Artificial Sequence
  synthetic hybrid polymerase
atgatcctggatgctgactacatcactgaagaaggcaaaccggttatccgtctgttcaaaaaagagaacggcgaatttaagattgagcatgatcgcacctttcgtccatacatttacgctctgctgaaagatgattctaagattgaggaagttaaaaaaatcactgctgagcgccatggcaagattgttcgtatcgttgatgcggaaaaggtagaaaagaaatttctgggcagaccaatcaccgtgtggagactgtatttcgaacatccacaagatgttccgactattcgcgagaaaattcgcgaacattctgcagttgttgacatcttcgaatacgatattccatttgcaaagcgttacctcatcgacaaaggcctgataccaatggagggcgatgaagaactcaagctcctggcgttcgctatagcaaccctctatcacgaaggcgaagagtttggtaaaggcccaattataatgatcagctatgcagatgaagaagaagcaaaggtgattacttggaaaaaaatagatctcccatacgttgaggttgtatcttccgagcgcgagatgattaagcgctttctcaaaattatccgcgagaaggatccggacattatcattacttataacggcgactcttttgacctcccatatctggcgaaacgcgcagaaaaactcggtattaaactgactatcggccgtgatggttccgagccgaagatgcagcgtatcggcgatatgaccgctgtagaagttaagggtcgtatccatttcgacctgtatcatgtaattcgtcgtactattaacctcccgacttacactctcgaggctgtatatgaagcaatttttggtaagccgaaggagaaggtatacgccgatgagattgcaaaggcgtgggaaaccggtgagggcctcgagcgtgttgcaaaatactccatggaagatgcaaaggcgacttatgaactcggcaaagaattcttcccaatggaagctcagctctctcgcctggttggccaaccactgtgggatgtttctcgttcttccaccggtaacctcgtagagtggtttctcctgcgcaaagcgtacgaacgcaacgaactggctccgaacaagccagatgaacgtgagtatgaacgccgtctccgcgagtcttacgctggtggctttgttaaagagccagaaaagggcctctgggaaaacatcgtgtccctcgattttcgcgctctgtatccgtctattatcattacccacaacgtgtctccggatactctcaaccgcgagggctgcagaaactatgatgttgctccggaagtaggccacaagttctgcaaggacttcccgggctttattccgtctctcctgaaacgtctgctcgatgaacgccaaaagattaagactaaaatgaaggcgtcccaggatccgattgaaaaaataatgctcgactatcgccaaagagcgattaaaatcctcgcaaactcttattacggctattatggctatgcaaaagcacgctggtactgtaaggagtgtgctgagtccgttactgcttggggtcgcgaatacatcgagttcgtgtggaaggagctcgaagaaaagtttggctttaaagttctctacattgacactgatggtctctatgcgactattccgggtggtaagtctgaggaaattaagaaaaaggctctagaatttgtggattacattaacgcgaagctcccgggtctcctggagctcgaatatgaaggcttttataaacgcggcttcttcgttaccaagaagaaatatgcgctgattgatgaagaaggcaaaattattactcgtggtctcgagattgtgcgccgtgattggagcgaaattgcgaaagaaactcaagctagagttctcgaggctattctcaaacacggcaacgttgaagaagctgtgagaattgtaaaagaagtaacccaaaagctctctaaatatgaaattccgccagagaagctcgcgatttatgagcagattactcgcccgctgcatgagtataaggcgattggtccgcacgtggctgttgcaaagagactggctgctaaaggcgtgaaaattaaaccgggtatggtaattggctacattgtactccgcggcgatggtccgattagcaaccgtgcaattctagctgaggaatacgatccgagaaagcacaagtatgacgcagaatattacattgagaaccaggtgctcccggcggtactccgtattctggagggttttggctaccgtaaggaagacctccgctggcaaaagactaaacagactggcctcacttcttggctcaacattaaaaaatccggtaccggcggtggcggtgcaaccgtaaagttcaagtacaaaggcgaagaaaaagaggtagacatctccaagatcaagaaagtatggcgtgtgggcaagatgatctccttcacctacgacgagggcggtggcaagaccggccgtggtgcggtaagcgaaaaggacgcgccgaaggagctgctgcagatgctggagaagcagaaaaagtga

SEQ ID NO: 4 - protein, 844 aa, Artificial Sequence
  synthetic hybrid polymerase
MILDADYITEEGKPVIRLFKKENGEFKIEHDRTFRPYIYALLKDDSKIEEVKKITAERHGKIVRIVDAEKVEKKFLGRPITVWRLYFEHPQDVPTIREKIREHSAVVDIFEYDIPFAKRYLIDKGLIPMEGDEELKLLAFAIATLYHEGEEFGKGPIIMISYADEEEAKVITWKKIDLPYVEVVSSEREMIKRFLKIIREKDPDIIITYNGDSFDLPYLAKRAEKLGIKLTIGRDGSEPKMQRIGDMTAVEVKGRIHFDLYHVIRRTINLPTYTLEAVYEAIFGKPKEKVYADEIAKAWETGEGLERVAKYSMEDAKATYELGKEFFPMEAQLSRLVGQPLWDVSRSSTGNLVEWFLLRKAYERNELAPNKPDEREYERRLRESYAGGFVKEPEKGLWENIVSLDFRALYPSIIITHNVSPDTLNREGCRNYDVAPEVGHKFCKDFPGFIPSLLKRLLDERQKIKTKMKASQDPIEKIMLDYRQRAIKILANSYYGYYGYAKARWYCKECAESVTAWGREYIEFVWKELEEKFGFKVLYIDTDGLYATIPGGKSEEIKKKALEFVDYINAKLPGLLELEYEGFYKRGFFVTKKKYALIDEEGKIITRGLEIVRRDWSEIAKETQARVLEAILKHGNVEEAVRIVKEVTQKLSKYEIPPEKLAIYEQITRPLHEYKAIGPHVAVAKRLAAKGVKIKPGMVIGYIVLRGDGPISNRAILAEEYDPRKHKYDAEYYIENQVLPAVLRILEGFGYRKEDLRWQKTKQTGLTSWLNIKKSGTGGGGATVKFKYKGEEKEVDISKIKKVWRVGKMISFTYDEGGGKTGRGAVSEKDAPKELLQMLEKQKK

SEQ ID NO: 5 - DNA, 23 bp, Artificial Sequence
  synthetic rpoB gene sequencing primer
tgcattttgtcatcaaccatgtg

SEQ ID NO: 6 - DNA, 17 bp, Artificial Sequence
  synthetic rpoB gene sequencing primer
ccttcacgtatgaacat

SEQ ID NO: 7 - DNA, 27 bp, Artificial Sequence
  synthetic Mycobacterium pirum forward primer
gaagatatgccgcatttagaagatggc

SEQ ID NO: 8 - DNA, 33 bp, Artificial Sequence
  synthetic Mycobacterium pirum reverse primer
caacaaccattgggtggtaaatcacaaaatggt

SEQ ID NO: 9 - DNA, 27 bp, Artificial Sequence
  synthetic Acholeplasma laidlawii forward primer
gaagatatgccatatttagcagatgga

SEQ ID NO: 10 - DNA, 33 bp, Artificial Sequence
  synthetic Acholeplasma laidlawii reverse primer
caacaaccaatgggtggtaaagctcaaaacggt

SEQ ID NO: 11 - DNA, 27 bp, Artificial Sequence
  synthetic Mycoplasma arginini forward primer
gaagaaatgccatacttagaagatgga

SEQ ID NO: 12 - DNA, 33 bp, Artificial Sequence
  synthetic Mycoplasma arginini reverse primer
caacaaccacttggtggtaaatcacaaaatggt

SEQ ID NO: 13 - DNA, 27 bp, Artificial Sequence
  synthetic Mycoplasma orale forward primer
gaagaaatgccttatttagaagatggt

SEQ ID NO: 14 - DNA, 33 bp, Artificial Sequence
  synthetic Mycoplasma orale reverse primer
caacaacctcttggtggtaaaagtcaaaacggt

SEQ ID NO: 15 - DNA, 27 bp, Artificial Sequence
  synthetic Mycoplasma salivarium forward primer
gaagatatgccttatttagaagatgga

SEQ ID NO: 16 - DNA, 33 bp, Artificial Sequence
  synthetic Mycoplasma salivarium reverse primer
caacaaccacttggtggaaagagtcaaaatggt

SEQ ID NO: 17 - DNA, 27 bp, Artificial Sequence
  synthetic Mycoplasma hominis forward primer
gaagaaatgccttatttagcagatgga

SEQ ID NO: 18 - DNA, 33 bp, Artificial Sequence; identical to SEQ ID NO: 16
  synthetic Mycoplasma hominis reverse primer

SEQ ID NO: 19 - DNA, 27 bp, Artificial Sequence
  synthetic Mycoplasma fermentans forward primer
gaagatatgccttatttagaagatggt

SEQ ID NO: 20 - DNA, 33 bp, Artificial Sequence
  synthetic Mycoplasma fermentans reverse primer
caacaacctcttggaggtaagagtcaaaacggt

SEQ ID NO: 21 - DNA, 27 bp, Artificial Sequence
  synthetic Mycoplasma hyorhinis forward primer
gaagatatgccatttttagaagatgga

SEQ ID NO: 22 - DNA, 33 bp, Artificial Sequence
  synthetic Mycoplasma hyorhinis reverse primer
caacaaccacttggaggaaaaagtcaaaacggt

SEQ ID NO: 23 - DNA, 26 bp, Artificial Sequence
  synthetic Forward primer consensus
gaagawatgccwtatttagaagatgg

SEQ ID NO: 24 - DNA, 32 bp, Artificial Sequence
  synthetic Reverse primer consensus
caacaaccactkggtggwaaragtcaaaaygg

SEQ ID NO: 25 - protein, 64 aa, Sulfolobus solfataricus
  DNA-binding protein 7d (Sso7d)
MATVKFKYKGEEKEVDISKIKKVWRVGKMISFTYDEGGGKTGRGAVSEKDAPKELLQMLEKQKK

SEQ ID NO: 26 - protein, 66 aa, Sulfolobus acidocaldarius
  DNA-binding protein 7d (Sac7d)
MVKVKFKYKGEEKEVDTSKIKKVWRVGKMVSFTYDDNGKTGRGAVSEKDAPKELLDMLARAEREKK

SEQ ID NO: 27 - protein, 65 aa, Sulfolobus acidocaldarius
  DNA-binding protein 7e (Sac7e)
MAKVRFKYKGEEKEVDTSKIKKVWRVGKMVSFTYDDNGKTGRGAVSEKDAPKELMDMLARAEKKK